(19) United States
(12) Patent Application Publication
(10) Pub. No.: US 2003/0008404 A1
(43) Pub. Date: Jan. 9, 2003

(54) Method of measuring an impurity profile of a semiconductor wafer and program for measuring an impurity profile of a semiconductor wafer

(76) Inventors: Mitsuhiro Tomita, Machida-shi (JP); Shoji Kozuka, Kawasaki-shi (JP); Tetsuya Tachibe, Kawasaki-shi (JP); Masamichi Suzuki, Yokohama-shi (JP)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(21) Appl. No.: 10/170,344

(22) Filed: Jun. 14, 2002

(30) Foreign Application Priority Data
Jun. 15, 2001 (JP) 2001-181790

(51) Int. Cl. G01N 33/00

(52) U.S. Cl. 436/72; 436/73; 436/77; 436/173; 436/183; 250/281; 250/282; 250/284

(57) ABSTRACT

Disclosed is a program for measuring the impurity in semiconductor wafer, comprising an instruction for supplying to a computer a reference dose, an instruction for causing the computer to convert each of a plurality of impurity profiles measured in a direction of depth of the semiconductor wafer into a dose, and an instruction for causing the computer to select a converted dose closest to the reference dose from the plurality of converted doses.

### CROSS-REFERENCE TO RELATED APPLICATIONS

This application is based upon and claims the benefit of priority from the prior Japanese Patent Application No. 2001-181790, filed Jun. 15, 2001, the entire contents of which are incorporated herein by reference.

### BACKGROUND OF THE INVENTION

1. Field of the Invention

The present invention relates to a method for determining a profile of impurities such as As and B measured in a direction of depth of a semiconductor wafer such as a silicon semiconductor wafer, a silicon film laminated on a silicon semiconductor wafer, or a silicon germanium film laminated on a silicon semiconductor wafer.

2. Description of the Related Art

A secondary ion mass spectrometric apparatus and a secondary ion mass spectrometry are known to the art as one of the surface analytical apparatuses and methods for measuring the impurity profile measured in a direction of depth of a semiconductor wafer.

In the secondary ion mass spectrometry, the surface of a sample arranged in a hermetically sealed container having a reduced pressure is continuously irradiated with primary ions, and the secondary ions generated by the sputtering excitation of the surface of the sample are subjected to a mass spectrometric analysis so as to analyze the concentration in the depth direction of the element present in the vicinity of the surface of the sample.

It is known to the art that, if the profile of the dopant impurity measured in the depth direction of a shallow diffusion layer of a silicon substrate sample is analyzed by the secondary ion mass spectrometry, the obtained profile is changed depending on the energy of the primary ions noted above. In other words, it is known to the art that the inclination in the profile is changed depending on the energy of the primary ions noted above. In this case, it is generally said that the obtained profile is rendered closer to the true profile, if the energy of the primary ion is rendered low.

However, where the B (boron) profile in the silicon semiconductor layer is measured by, for example, method 1) or method 2) given below, the profile of the B concentration in a region not deeper than 5 nm in the case of employing method 1) is rendered widely different from that in the case of employing method 2) even in the case of using primary ions having a low energy not higher than, for example, 1 keV, giving rise to the problem that it is difficult to judge which profile represents the true profile or is close to the true profile, or that both of profiles are not the true profile:

1) The method in which B⁺ ions are measured by using oxygen primary ions (O₂⁺) having a low energy not higher than, for example, 1 keV and having an incident angle (angle between the passageway of the oxygen primary ions and the line normal to the surface of the sample) of 0° to 20°, and the B profile measured in the depth direction is obtained from the B⁺ secondary ions profile measured in the depth direction.

2) The method in which the B⁺ secondary ions are measured by using oxygen primary ions (O₂) while blowing an oxygen gas against the surface of the sample, the oxygen primary ions having a low energy not higher than, for example, 1 keV and having an incident angle (angle between the passageway of the oxygen primary ions and the line normal to the surface of the sample) of 60° to 45°, and the B profile measured in the depth direction is obtained from the B⁺ secondary ions profile measured in the depth direction.

Also, where the As (arsenic) profile in a silicon semiconductor layer is measured by, for example, method 3) or method 4) given below, the profile of the As concentration in a surface region not deeper than 5 nm, particularly, the As concentration in the vicinity of the native oxide formed on the surface of the silicon sample, in the case of employing method 3) widely differs from that in the case of employing method 4), giving rise to the problem that it is impossible to judge which profile represents the true profile or close to the true profile, or that both of profiles are not the true profile:

3) The AsSi⁻ and Si₂⁻ secondary ions are measured by using Cs primary ions (Cs⁺) having a low energy not higher than, for example, 1 keV and having an incident angle (angle between the passageway of the Cs primary ions and a line normal to the surface of the sample) of 70° to 50°, and the As profile measured in the depth direction is obtained from the AsSi⁻/Si₂⁻ ion intensity ratio in each depth (analytical point in each depth).

4) The AsSi⁻ and Si₂⁻ secondary ions are measured by using Cs primary ions (Cs⁺) having a low energy not higher than, for example, 1 keV and having an incident angle (angle between the passageway of the Cs primary ions and a line normal to the surface of the sample) of 70° to 50°, and As profile measured in the depth direction is obtained from the distribution in the depth direction of the average Si₂⁻ ion intensity ratio (AsSi⁻/Average Si₂⁻) in a deep region where the Si₂⁻ ion intensity is rendered constant, i.e., the region deeper than 5 nm.

As described above, if the measuring conditions such as the kind of the secondary ion used for calculating the intensity ratio are changed in using the secondary ion mass spectrometric apparatus, the B profile measured in the depth direction and the As profile measured in the depth direction are changed, making it very difficult to determine which profile in the depth direction, i.e., the profile obtained by which method, represents the true profile or a profile close to the true profile.

### BRIEF SUMMARY OF THE INVENTION

An object of the present invention is to provide a method of measuring the impurities profile in a semiconductor wafer, which permits determining which of the impurities profiles measured in the depth direction obtained by a plurality of methods represents the true profile or a profile close to the true profile.

Another object of the present invention is to provide a program for measuring the impurities profile in a semiconductor wafer, which permits determining which of the impurities profiles measured in the depth direction obtained by a plurality of methods represents the true profile or a profile close to the true profile.

Another object of the present invention is to provide a method of measuring the impurities profile in a semiconductor wafer, which permits obtaining a true profile or a profile close to the true profile.

Further, still another of the present invention is to provide a program for measuring the impurities profile in a semiconductor wafer, which permits obtaining a true profile or a profile close to the true profile.

According to a first aspect of the present invention, there is provided a method of measuring an impurity profile of a semiconductor wafer, comprising:

obtaining a reference dose by measuring a dose of an impurity in a semiconductor wafer by one measuring method selected from the group consisting of a chemical analysis, a nuclear reaction analysis, a Rutherford backscattering spectrometry and a particle induced X-ray emission analysis, the measuring method being selected in accordance with the kind of the impurity;

obtaining a plurality of impurity profiles measured in a direction of depth of the semiconductor wafer by a plurality of measuring methods;

obtaining a plurality of converted doses by converting each of the plurality of impurity profiles measured in the direction of depth into a dose;

selecting a converted dose closest to the reference dose from the plurality of converted doses; and

selecting an impurity profile which gives the selected dose from the plurality of impurity profiles measured in the direction of depth, as the impurity profile of the semiconductor.

According to a second aspect of the present invention, there is provided a method of measuring an impurity profile of a semiconductor wafer, comprising:

obtaining a reference dose by measuring a dose of an impurity in a semiconductor wafer by one measuring method selected from the group consisting of a chemical analysis, a nuclear reaction analysis, a Rutherford backscattering spectrometry and a particle induced X-ray emission analysis, the measuring method being selected in accordance with the kind of the impurity;

obtaining a plurality of impurity profiles measured in a direction of depth of the semiconductor wafer by a plurality of measuring methods;

obtaining a plurality of converted doses by converting each of the plurality of impurity profiles measured in the direction of depth into a dose;

selecting a converted dose closest to the reference dose from the plurality of converted doses;

calculating a value X in formula (1) given below:

X=(a−b)/a (1)

where “a” is the reference dose (atoms/cm²), and “b” is the selected dose (atoms/cm²); and

selecting an impurity profile which gives a selected dose that permits the value of X to fall within a range from −0.1 to 0.1 from the plurality of impurity profiles measured in the direction of depth, as the impurity profile of the semiconductor.

According to a third aspect of the present invention, there is provided a method of measuring an impurity profile of a semiconductor wafer, comprising:

obtaining a reference dose by measuring a dose of an impurity in a semiconductor wafer by one measuring method selected from the group consisting of a chemical analysis, a nuclear reaction analysis, a Rutherford backscattering spectrometry and a particle induced X-ray emission analysis, the measuring method being selected in accordance with the kind of the impurity; and

obtaining an impurity profile measured in a direction of depth of the semiconductor wafer, the impurity profile giving a converted dose that satisfies formula (2) given below:

−0.1≦{(a−b)/a}≦0.1 (2)

where “a” is the reference dose (atoms/cm²), and “b” is a converted dose (atoms/cm²) obtained by converting an impurity profile measured in the direction of depth into a dose.

According to a fourth aspect of the present invention, there is provided a program for measuring an impurity profile of a semiconductor wafer, comprising:

an instruction for supplying to a computer as a reference dose at least one kind of the dose selected from the group consisting of a dose of an impurity in a semiconductor wafer obtained by a chemical analysis, a dose of the impurity in the semiconductor wafer obtained by a nuclear reaction analysis, a dose of the impurity in the semiconductor wafer obtained by a Rutherford backscattering spectrometry, and a dose of the impurity in the semiconductor wafer obtained by a particle induced X-ray emission analysis;

an instruction for causing the computer to convert each of a plurality of impurity profiles measured in a direction of depth of the semiconductor wafer into a dose, the plurality of impurity profiles being obtained by plural methods; and

an instruction for causing the computer to select a converted dose closest to the reference dose from the plurality of converted doses.

According to a fifth aspect of the present invention, there is provided a program for measuring an impurity profile of a semiconductor wafer, comprising:

an instruction for supplying to a computer as a reference dose at least one kind of the dose selected from the group consisting of a dose of an impurity in a semiconductor wafer obtained by a chemical analysis, a dose of the impurity in the semiconductor wafer obtained by a nuclear reaction analysis, a dose of the impurity in the semiconductor wafer obtained by a Rutherford backscattering spectrometry, and a dose of the impurity in the semiconductor wafer obtained by a particle induced X-ray emission analysis;

an instruction for causing the computer to convert an impurity profile measured in a direction of depth of the semiconductor wafer into a dose; and

an instruction for causing the computer to calculate a value X in formula (5) given below:

X=(a−b)/a (5)

where “a” is the reference dose (atoms/cm²), and “b” is the converted dose (atoms/cm²); and

an instruction for causing the computer to find an impurity profile which gives a converted dose that allows the value of X to fall within a range from −0.1 to 0.1.

### BRIEF DESCRIPTION OF THE SEVERAL VIEWS OF THE DRAWING

FIG. 1 is a flow chart exemplifying a method of the present invention for measuring an impurity profile in a semiconductor wafer;

FIG. 2 schematically shows the construction of a secondary ion mass spectrometric apparatus used in Example 1 for measuring the impurity profile measured in the depth direction of a semiconductor wafer;

FIG. 3 is a graph showing the As concentration profile measured in the depth direction of a semiconductor wafer, said profile being obtained by the secondary ion mass spectrometry in Example 1;

FIG. 4 is a flow chart exemplifying a program for measuring an impurity profile in the depth direction of a semiconductor wafer in Example 1;

FIG. 5 is a graph showing the B concentration profile measured in the depth direction of a semiconductor wafer, said profile being obtained by the secondary ion mass spectrometry in Example 2;

FIG. 6 is a graph showing the Sb concentration profile measured in the depth direction of a semiconductor wafer, said profile being obtained by the secondary ion mass spectrometry in Example 4; and

FIG. 7 is a graph showing the In concentration profile measured in the depth direction of a semiconductor wafer, said profile being obtained by the secondary ion mass spectrometry in Example 5.

### DETAILED DESCRIPTION OF THE INVENTION

An example of the method of the present invention for measuring the impurity concentration in a semiconductor wafer and an example of the program of the present invention for measuring the impurity concentration in a semiconductor wafer will now be described.

A first method of the present invention for measuring an impurity profile of a semiconductor wafer comprises:

obtaining a reference dose by measuring the dose of an impurity in a semiconductor wafer by one measuring method selected from the group consisting of a chemical analysis, a nuclear reaction analysis, a Rutherford backscattering spectrometry and a particle induced X-ray emission analysis, the measuring method being selected in accordance with the kind of the impurity;

obtaining a plurality of impurity profiles measured in a direction of depth of the semiconductor wafer by a plurality of measuring methods;

obtaining a plurality of converted doses by converting each of the plurality of impurity profiles measured in the direction of depth into a dose;

selecting a converted dose closest to the reference dose from the plurality of converted doses; and

selecting an impurity profile which gives the selected dose from the plurality of impurity profiles measured in the direction of depth, as the impurity profile of the semiconductor.

A second method of the present invention for measuring an impurity profile of a semiconductor wafer comprises:

obtaining a reference dose by measuring a dose of an impurity in a semiconductor wafer by one measuring method selected from the group consisting of a chemical analysis, a nuclear reaction analysis, a Rutherford backscattering spectrometry and a particle induced X-ray emission analysis, the measuring method being selected in accordance with the kind of the impurity;

obtaining a plurality of impurity profiles measured in a direction of depth of the semiconductor wafer by a plurality of measuring methods;

obtaining a plurality of converted doses by converting each of the plurality of impurity profiles measured in the direction of depth into a dose;

selecting a converted dose closest to the reference dose from the plurality of converted doses;

calculating a value X in formula (1) given below:

X=(a−b)/a (1)

where “a” is the reference dose (atoms/cm²), and “b” is the selected dose (atoms/cm²); and

selecting an impurity profile which gives a selected dose that permits the value of X to fall within a range from −0.1 to 0.1 from the plurality of impurity profiles measured in the direction of depth, as the impurity profile of the semiconductor.

A third method of the present invention for measuring the distribution of the impurity concentration in the depth direction of a semiconductor wafer comprises:

obtaining a reference dose by measuring a dose of an impurity in a semiconductor wafer by one measuring method selected from the group consisting of a chemical analysis, a nuclear reaction analysis, a Rutherford backscattering spectrometry and a particle induced X-ray emission analysis, the measuring method being selected in accordance with the kind of the impurity; and

obtaining an impurity profile measured in a direction of depth of the semiconductor wafer, the impurity profile giving a converted dose that satisfies formula (2) given below:

−0.1≦{(a−b)/a}≦0.1 (2)

where “a” is the reference dose (atoms/cm²), and “b” is a converted dose (atoms/cm²) obtained by converting an impurity profile measured in the direction of depth into a dose.

It should be noted that the term “dose” represents the value calculated by X/S, where X denotes the total amount of the impurity atoms in a semiconductor wafer, and S denotes the area of the semiconductor wafer.

It is possible to measure simultaneously the reference dose and a plurality of impurity profiles measured in the depth direction. Alternatively, it is also possible to measure a plurality of impurity profiles measured in the depth direction either before or after the measurement of the reference dose.

The method capable of measuring with a high accuracy and with a high precision the reference dose of the impurity in a semiconductor wafer differs depending on the kind of the impurity. Therefore, it is desirable to measure the reference dose by a suitable method selected from among the four kinds of the methods described previously in accordance with the kind of the impurity.

A first program of the present invention for measuring an impurity profile of a semiconductor wafer comprises:

an instruction for supplying to a computer as a reference dose at least one kind of a dose selected from the group consisting of a dose of an impurity in a semiconductor wafer obtained by a chemical analysis, a dose of the impurity in the semiconductor wafer obtained by a nuclear reaction analysis, a dose of the impurity in the semiconductor wafer obtained by a Rutherford backscattering spectrometry, and a dose of the impurity in the semiconductor wafer obtained by a particle induced X-ray emission analysis;

an instruction for causing the computer to convert each of a plurality of impurity profiles measured in a direction of depth of the semiconductor wafer into a dose, the plurality of impurity profiles being obtained by plural methods; and

an instruction for causing the computer to select a converted dose closest to the reference dose from the plurality of converted doses.

It is desirable for the first program of the present invention for measuring the impurity concentration in a semiconductor wafer to further comprising:

an instruction for causing the computer to calculate the value of X in formula (3) given below:

X=(a−b)/a (3)

where “a” represents the reference dose (atoms/cm²), and “b” represents the selected dose (atoms/cm²); and

an instruction for causing the computer to select an impurity profile which gives a selected dose that permits the value of X to fall within a range from −0.1 to 0.1, from the plurality of impurity profiles measured in the direction of depth.

A second program of the present invention for measuring an impurity profile of a semiconductor wafer comprises:

an instruction for supplying to a computer as a reference dose at least one kind of a dose selected from the group consisting of a dose of an impurity in a semiconductor wafer obtained by a chemical analysis, a dose of the impurity in the semiconductor wafer obtained by a nuclear reaction analysis, a dose of the impurity in the semiconductor wafer obtained by a Rutherford backscattering spectrometry, and a dose of the impurity in the semiconductor wafer obtained by a particle induced X-ray emission analysis;

an instruction for causing the computer to convert an impurity profile measured in a direction of depth of the semiconductor wafer into a dose; and

an instruction for causing the computer to calculate a value X in formula (4) given below:

X=(a−b)/a (4)

where “a” is the reference dose (atoms/cm²), and “b” is the converted dose (atoms/cm²); and

an instruction for causing the computer to find an impurity profile which gives a converted dose that allows the value of X to fall within a range from −0.1 to 0.1.

The present invention is mainly intended to provide a method of obtaining the correct profile of the impurity concentration in the depth direction of a semiconductor wafer. In the method of the present invention, the absolute amount of the impurity in a semiconductor wafer is measured first by a method that permits the measurement with a high accuracy and a high precision, the method being selected from the group consisting of a chemical analysis, a nuclear reaction analysis, a Rutherford backscattering spectrometry, and a particle induced X-ray emission analysis, so as to calculate the dose of the impurity in the semiconductor wafer, followed by selecting an impurity profile from a plurality of impurity profiles in the depth direction by using the obtained impurity dose. Incidentally, in the present invention, it is desirable for the impurity profile measured in the planar direction of a semiconductor wafer to be uniform or substantially uniform, i.e., the concentration fluctuation of +10%.

It should be noted that, in the stage of the research and development of a silicon semiconductor device, if the impurity profile measured in the depth direction of a silicon semiconductor wafer after a plurality of manufacturing steps such as a plurality of ion implantation steps and a plurality of heat treatment steps is measured by the technology giving the impurity profile in the depth direction, which is selected or obtained by the present invention, the obtained impurity profile in the depth direction exhibits the true profile or the profile close to the true profile. Therefore, it is possible to determine precisely which manufacturing process of a silicon semiconductor device is most excellent by comparing and studying the shape of the profile, the impurity dose obtained therefrom, etc. In other words, it is possible to compare and study aptly a plurality of manufacturing steps in the stage of the research and development of a silicon semiconductor device so as to make it possible to carry out the research and development efficiently.

It should also be noted that, if the impurity profile measured in the depth direction in the factory of a silicon semiconductor device is measured by the technology giving the impurity profile that is judged to be the true profile in the present invention, it is possible to compare and study accurately the shape in the impurity profile in the depth direction, the impurity dose obtained therefrom, etc. in respect of a good sample and a defective sample so as to make it possible to perform aptly the defective analysis of a silicon semiconductor device derived from the abnormality in the profile and dose.

The impurity profile measured in the depth direction can be measured by any surface analytical method as far as it is possible to obtain the element profile measured in the depth direction. However, it is desirable to employ a secondary ion mass spectrometry or a sputtered neutral mass spectrometry.

Where the impurity comprises at least one element selected from the group consisting of As, P, Sb, B, In, Ge and Ga, it is desirable for the reference dose to be measured by a chemical analysis and for the plurality of impurity profiles measured in the depth direction to be obtained by a plurality of technologies owned by the secondary ion mass spectrometric apparatus. By the particular construction, it is possible to improve the reliability in respect of the determine as to whether or not the obtained impurity profile represents the true profile.

The chemical analysis implies a method of obtaining the composition of a solid sample or the concentration of the impurity contained in the solid sample by dissolving the solid sample in a solvent to obtain a sample solution and measuring the concentration of the aimed element contained in the sample solution.

It is possible to use, for example, a hydrofluoric acid solution, a nitric acid solution, a hydrochloric acid solution, a sulfuric acid solution, a perchloric acid solution, a hydrogen peroxide solution or a mixed solution thereof as the solvent for dissolving the solid sample.

For the composition analysis or the measurement of the concentration of the impurity element in respect of the sample solution having the solid sample dissolved therein, it is possible to employ, for example, an inductively coupled plasma mass spectroscope, an inductively coupled plasma emission spectroscope, an atomic absorption spectroscope, an absorptiometric apparatus, a titration analytical method or a weight analytical method.

Also, it is possible to separate the component or element contained in the sample solution and obstructing the measurement by, for example, the ion exchange method or the evaporation drying method before the sample solution is supplied to the measuring machine.

It is possible to employ any type of the chemical analysis, as far as the total amount of the impurities contained in the semiconductor wafer such as As, P, Sb, B, In, Ge and Ga can be analyzed accurately. However, where the arsenic (As) concentration is to be measured, it is desirable to carry out the chemical analysis by the method described below. Specifically, the chemical analysis for this purpose comprises decomposing a semiconductor wafer by using nitric acid, hydrofluoric acid and at least one kind of the compound selected from the group consisting of potassium permanganate, potassium periodate and periodic acid so as to obtain a decomposed liquid containing arsenic and at least one element selected from the group consisting of manganese, potassium and iodine;

ionizing the at least one element contained in the decomposed liquid;

removing the ions of the at least one element from the decomposed liquid by using an ion exchange resin; and

subjecting arsenic contained in the decomposed liquid to a mass spectrometry.

Also, where the indium (In) concentration is to be measured, it is desirable to carry out the chemical analysis by the method described below. Specifically, the chemical analysis for this purpose comprises dissolving a semiconductor wafer by using a mixed solution containing hydrofluoric acid and nitric acid so as to obtain a decomposed liquid containing In, and subjecting indium (In) in the decomposed liquid to a mass spectrometry.

On the other hand, it is desirable to convert the total amount of the impurities (e.g., As, P, Sb, B, In, Ge, Ga) contained in a semiconductor wafer, which is obtained by the chemical analysis, into the dose as follows. In order to evaluate accurately the surface area of the semiconductor wafer, it is desirable to cut out the sample in a square or rectangular shape. Where the sample is not square nor rectangular, it is possible to calculate the surface area from the weight and the thickness of the sample. It is possible to calculate the dose of the impurity (e.g., As, P, Sb, B, In, Ge, Ga) contained in a semiconductor wafer by using the total amount of the impurity (e.g., As, P, Sb, B, In, Ge, Ga) contained in a semiconductor wafer thus obtained and the surface area of the semiconductor wafer in accordance with formula (A) given below:

C=W/S (A)

where C represents the dose of the impurity element contained in the semiconductor wafer, W represents the total amount of the impurity elements contained in the semiconductor wafer, and S represents the surface area of the semiconductor wafer.

Where the impurity includes at least one element selected from the group consisting of B and F, it is desirable to measure the reference dose by the nuclear reaction analysis and to obtain a plurality of impurity profiles in the depth direction by a plurality of methods each involving a secondary ion mass spectrometric apparatus. The particular construction makes it possible to further improve the reliability of the determination as to whether or not the measured the impurity profile represents the true profile.

The nuclear reaction analysis implies the method of measuring the amount of the impurity in a semiconductor wafer by irradiating the semiconductor wafer with particles accelerated to have a high energy, e.g., a high energy not lower than 0.5 MeV, so as to bring about a nuclear reaction between the accelerated particles and the impurity contained in the semiconductor wafer and by measuring the amount of the radiation generated by the nuclear reaction so as to measure the amount of the impurity contained in the semiconductor wafer. The nuclear reaction analysis employed in the present invention is not particularly limited as far as the amount of the impurity contained in a semiconductor wafer is measured by the method described above. The accelerated particles used for irradiating the semiconductor wafer generally consist of ions. However, it is possible for the accelerated particles to be formed of a neutron flux such that a nuclear reaction is brought about between the neutron flux and the impurity in the semiconductor wafer so as to measure the amount of the impurity contained in the semiconductor wafer by detecting the amount of the radiation formed by the nuclear reaction. In this case, the impurity to be measured is not limited to B and F. It also possible for any of As, P, Sb, B, In, Ge, F and Ga to be used as the impurity to be measured.

In the case of measuring B contained in the semiconductor wafer, it is desirable for the accelerated particles to be hydrogen ions (protons) having an energy of 0.65 MeV and for the radiation formed by the nuclear reaction and detected to be an α particle. Also, in the case of measuring F contained in the semiconductor wafer, it is desirable for the accelerated particles to be hydrogen ions (protons) having an energy range from 0.872 MeV to 0.9 MeV and for the radiation formed by the nuclear reaction and detected to be a γ particle or an α particle.

Where the impurity to be measured is at least one kind of an element selected from the group consisting of As, Sb, In, Ge and Ga, it is desirable for the reference dose to be measured by the Rutherford backscattering spectrometry or the chemical analysis and for the plurality of impurity profiles measured in the depth direction to be obtained by a plurality of methods owned by the secondary ion mass spectrometric apparatus. By the particular construction, it is possible to further improve the reliability in respect of the determination as to whether or not the obtained impurity profile represents the true profile. in the Rutherford backscattering spectrometry, a semiconductor wafer is irradiated with particles accelerated to have a high energy, e.g., 0.3 MeV to 5 MeV, and the accelerated particles are scattered into the impurity contained in the semiconductor wafer so as to be released from within the semiconductor wafer. Then, the amount of the impurity contained in the semiconductor wafer and the depth of the position where the impurity was present in the semiconductor wafer are measured by detecting the energy and the amount of the backscattered particles. The Rutherford backscattering spectrometry employed in the present invention is not particularly limited as far as the amount of the impurity contained in the semiconductor wafer and the depth of the position where the impurity was present in the semiconductor wafer are measured as described above. The energy and amount of the backscattered particles are detected by a semiconductor detector. It is possible to measure the energy through a magnetic field type or an electric field type energy analyzer. In general, He ions and H ions (protons) are used as the accelerated particles irradiating the sample, which are also the particles that are backscattered. However, it is possible to use any kind of ions as the accelerating particles as far as the accelerating particle is lighter than the impurity atom (e.g., As, Sb, In, Ge, Ga) contained in the semiconductor wafer to be measured.

Where the impurity includes at least one kind of an element selected from the group consisting of As, Ge, Sb, In and Ga, it is desirable for the reference dose to be measured by the particle induced X-ray emission analysis and for the plurality of impurity profiles measured in the depth direction to be obtained by a plurality of methods owned by the secondary ion mass spectrometric apparatus. By the particular construction, it is possible to further improve the reliability in respect of the determination as to whether or not the obtained impurity profile represents the true profile.

In the particle induced X-ray emission analysis, a semiconductor wafer is irradiated with particles accelerated to have a high energy (e.g., 0.1 MeV to 10 MeV), and the amount of the impurity contained in the semiconductor wafer is measured by detecting the energy and the intensity of the characteristic X-ray generated by the mutual function between the accelerated particles and the atoms constituting the semiconductor wafer. The particle induced X-ray emission analysis employed in the present invention is not particularly limited as far as the amount of the impurity contained in the semiconductor wafer is measured as described above. The energy and amount of the radiated X-ray are detected by an energy scattering type X-ray detector or a wavelength scattering type X-ray detector. In general, He ions and H ions (protons) are used as the accelerated particles irradiating the sample. However, it is also possible to use ions having a larger mass.

The secondary ion mass spectrometric apparatus used for the secondary ion mass spectrometry comprises a hermetic container arranged under a vacuum environment and capable of having a sample disposed thereon, an ion generator which irradiates a part of the sample with the primary ion beam, and an analyzer which analyzes the secondary ions excited by the primary ion beam so as to be sputtered from the surface of the sample.

It is possible to use any kind of an ion as the primary ion, though it is desirable to use at least one kind of an ion selected from the group consisting of Cs⁺, O₂⁺, O⁻, Ar⁺, Kr⁺, Xe⁺, Ga⁺ and In⁺. Also, it is desirable for the energy of the primary ion to be not higher than 1 keV.

Any kind of the analyzer which analyzes the secondary ion can be used in the present invention, as far as the analyzer is capable of the mass separation of the secondary ion and also capable of detecting the amount of the mass-separated secondary ions. However, it is desirable for a double focusing mass spectrometer, a quadrupole mass spectroscopy, or a time-of-flight type mass spectrometer to be used as the mass separator. Where the time-of-flight type mass spectrometer is used as the mass analyzer, it is possible to use alternately two kinds of ions consisting of at least one first ion selected from the group consisting of Cs⁺, O₂⁺ and O⁻ and at least one second ion selected from the group consisting of Ar⁺, Kr⁺, Xe⁺, Ga⁺ and In⁺. The ions selected from the group consisting of Cs⁺, O₂⁺ and O⁻ are used for the sputtering of the sample surface in the depth direction. On the other hand, the ions selected from the group consisting of Ar⁺, Kr⁺, Xe⁺, Ga⁺ and In⁺ are used for detecting the secondary ions. Further, it is desirable for the secondary ion mass spectrometric apparatus to be equipped with a nozzle for blowing an oxygen gas against the surface of the sample during the analysis.

The secondary neutral particle mass spectroscope used for the secondary neutral particle mass spectrometry comprises a hermetic container arranged under a vacuum environment and capable of having a sample mounted therein, an ion generating mechanism which irradiates a part of the sample with a primary ion beam, a laser or electron beam irradiating apparatus which ionizes the neutral particles excited by the primary ion beam so as to be sputtered from the surface of the sample, and an analyzing mechanism which analyzes the particles ionized by the laser or electron beam irradiating apparatus.

Any kind of the ion can be used as the primary ion, though it is desirable for the primary ion to include at least one kind of an ion selected from the group consisting of Ar⁺, Kr⁺, Xe⁺, Ga⁺ and In⁺. Also, it is desirable for the primary ion to have an energy not higher than 1 keV. The analyzing mechanism which analyzes the particles ionized by the laser or electron beam irradiating apparatus, which is used in the present invention, is not particularly limited, as far as the analyzing mechanism is capable of the mass separation and also capable of detecting the amount of the mass-separated particles, though it is desirable to use a double focusing mass spectrometer, a quadrupole mass spectroscopy or a time-of-flight type mass spectrometer as the mass separator.

In the secondary ion mass spectrometry, it is desirable to obtain a plurality of impurity profiles measured in the depth direction by changing at least one of the measuring conditions including the method for converting the number of counts of the secondary ions into the impurity concentration (atoms/cm³), the kind of the primary ion, the energy of the primary ion, the incident angle of the primary ion, and the kind of the secondary ion every time the impurity profile in the depth direction is measured.

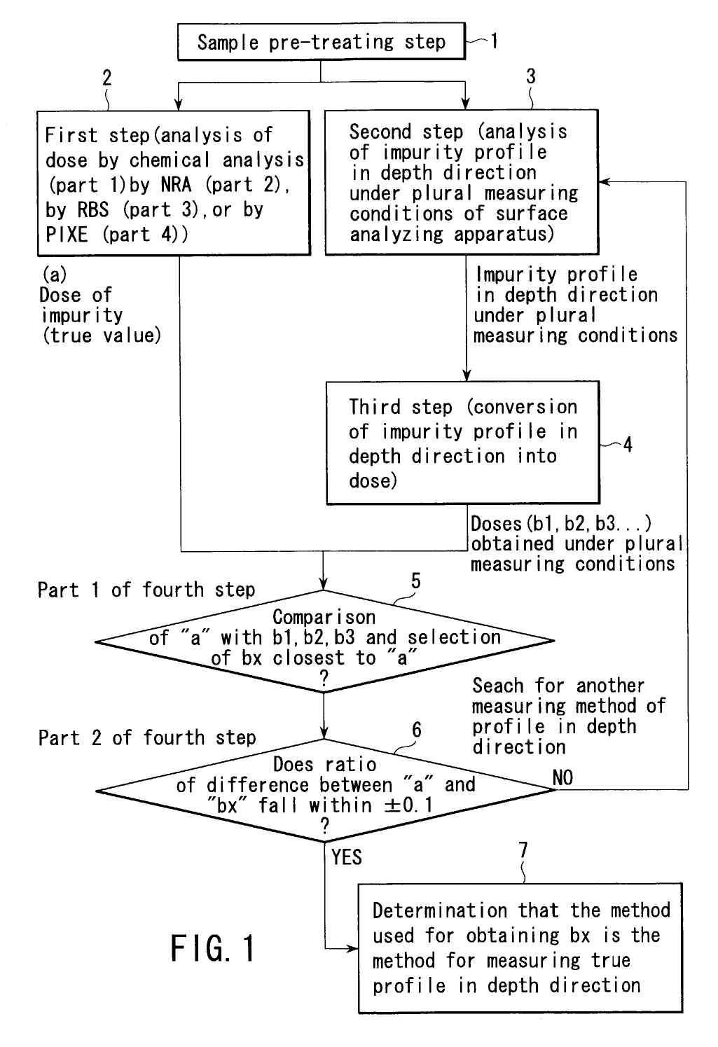

The method of the present invention for measuring the impurity profile of a semiconductor wafer will now be described with reference to the flow chart shown in FIG. 1. The method of measuring impurity profiles measured in the depth direction, which uses as the reference dose the dose of the impurity in a semiconductor wafer determined by the chemical analysis, will now be described as a typical example.

The pre-treating step of the sample, i.e., process 1 shown in FIG. 1, will now be described first.

(Sample Pre-Treating Step 1)

In this step, prepared is a sample of a semiconductor wafer to be analyzed for measuring the impurity profile in the depth direction.

In general, a semiconductor wafer has a size of, for example, 6 inches or 8 inches, which is too large for the semiconductor wafer to be analyzed by a series of steps (the chemical analysis, the Rutherford backscattering spectrometry, the nuclear reaction analysis, the particle induced X-ray emission analysis, the secondary ion mass spectrometry, the secondary neutral particle mass spectrometry). Also, the analysis is applied to an optional one point. Therefore, it is desirable to cut out a piece sized 3 cm×3 cm from the semiconductor wafer. For cutting the semiconductor wafer, it is possible to use, for example, a diamond cutter. Also, in order to cut out a piece of the semiconductor wafer accurately in the square or rectangular shape, it is desirable to use a sample cutting device such as a dicing saw. Also, since stains such as the cutting dust are attached to the sample cut out of the semiconductor wafer, it is desirable to remove the cutting dust from the sample by the air blow or to wash the sample with a pure water or an organic solvent such as an alcohol series organic solvent or a ketone series organic solvent.

Particularly, in evaluating the impurity dose in a semiconductor wafer by a chemical analysis, it is necessary to evaluate accurately the surface area of the semiconductor wafer. Therefore, it is desirable to cut out the sample in a square or rectangular shape. It is desirable to calculate the area of the semiconductor wafer cut out in a square or rectangular shape by accurately measuring the lengths of the vertical side and the lateral side of the semiconductor wafer with, for example, a slide gage or a micrometer, followed by calculating the area of the sample by multiplying the measured length of the vertical side by the measured length of the lateral side. Also, it is possible to calculate the area of the sample from the weight of the sample, in the case where the sample cut out of the semiconductor wafer is not shaped square or rectangular.

Part 1 of the first step included in process 2 shown in FIG. 1 will now be described.

(Part 1 of First Step)

The method of obtaining the impurity dose in a semiconductor wafer by a chemical analysis will now be described, with the method of obtaining the As dose in a silicon semiconductor layer taken up as an example.

(First Reaction Step)

A silicon semiconductor wafer sample is successively brought into contact with a potassium permanganate solution, nitric acid, and hydrofluoric acid so as to dissolve the sample in the solution. In this case, it is possible to confirm the progress of the reaction until the reaction is finished by visually recognizing the color (pink) of the potassium permanganate solution. Where the pink color cannot be recognized and, thus, the reaction is not finished, the potassium permanganate solution is added. By performing the particular operation, it is possible to prevent As from forming a compound with silicon so as to be evaporated.

Then, the obtained sample solution is condensed by heating, thereby finishing the first reaction step to obtain a decomposed liquid containing As.

The chemical reaction in the first reaction step can be represented by formula (1) given below. Also, it is possible to use at least one reagent selected from the group consisting of a potassium periodate solution and a periodic acid solution in place of the potassium permanganate solution because the reaction equal to that in the case of using the potassium permanganate solution takes place. Where a potassium periodate solution or a periodic acid solution is used in place of the potassium permanganate solution, the chemical reaction carried out in the first reaction step is represented by formula (2) given below:

2MnO₄⁻+16H⁺+5As→2Mn²⁺+5As⁵⁺+8H₂O (1)

2IO₃⁻+12H⁺+5As³⁺→2I⁺+5As⁵⁺+6H₂O (2)

In the first reaction step, the valency of As is increased from the tri-valency state to the penta-valency state under which As is unlikely to form a chemical substance that is likely to be scattered. Also, silicon is converted in this step into a compound that is likely to be scattered such as H₂SiF₆ so as to be removed, though silicon is not included in the reaction formulas given above.

In the first reaction step, it is desirable to use a container made of a material capable of resisting chemicals because nitric acid and hydrofluoric acid are used in the first reaction step. For example, it is desirable to use a container made of a fluorocarbon polymers. Further, in view of the subsequent operability, it is desirable to use a bottle made of a fluorocarbon polymers and having an inner volume of about 100 to 250 ml. It should also be noted that the concentration of the reagent required for decomposing the wafer in the first reaction step is not particularly limited, as far as the sample can be decomposed. However, it is desirable to use a reagent of a high purity, which is low in the impurity content. Particularly, it is desirable for the concentration of potassium permanganate to be 4 to 6%, for the concentration of nitric acid to be 50 to 68%, and for the concentration of hydrofluoric acid to be 30 to 38% in view of the decomposition of the sample. If the concentrations of the reagents fail to fall within the ranges noted above, the decomposition of the sample tends to be rendered difficult. When it comes to the acid concentration, the decomposing time of the wafer tends to be shortened with increase in the acid concentration and, thus, it is desirable for the acid concentration to be high.

(Second Reaction Step)

The sample solution after the first reaction step is dissolved again by using nitric acid and hydrogen peroxide so as to finish the second reaction step in which a sample solution containing As can be obtained.

In this step, nitric acid and hydrogen peroxide are added to the sample solution so as to permit the elements constituting the reagent except the element to be analyzed (As), e.g., manganese, potassium and iodine, to react with nitric acid and hydrogen peroxide, thereby converting manganese, potassium and iodine into cations or anions. The elements that are ionized are changed depending on the reagent used in the first reaction step. In this case, at least one element selected from the group consisting of manganese, potassium and iodine is ionized. Where potassium permanganate is used in the first reaction step, the chemical reaction carried out in the second reaction step is represented by formula (3) given below. On the other hand, in the case of using a potassium periodate solution in the first reaction step, the chemical reaction carried out in the second reaction step is represented by formula (4) given below:

5H₂O₂+MnO₄⁻+6H⁺→2Mn²⁺+2H₂O+5O₂ (3)

5H₂O₂+2IO₃⁻+6H⁺→I₂⁺+8H₂O+4O₂ (4)

Manganese, potassium and iodine are ionized in the second reaction step so as to be prepared for the subsequent adsorption using an ion exchange resin.

In the re-dissolving method in the second reaction step, it is desirable to use the reagent (nitric acid, hydrogen peroxide) of a high purity, which is low in the impurity content. It is desirable for the concentration of the hydrogen peroxide solution to be low because hydrogen peroxide tends to be scattered. However, it is desirable for the hydrogen peroxide concentration to fall within a range of between 20 and 35% in order to facilitate the subsequent ion exchange separating operation. On the other hand, it is desirable for the nitric acid concentration to be high in order to decompose MnO₄⁻ promptly. However, it is desirable for the nitric acid concentration to fall within a range of between 50 and 60% in order to facilitate the subsequent ion exchange separating operation.

(Ion Exchange Reaction Step)

After the second reaction step, the sample solution noted above is brought into contact with an ion exchange resin so as to allow the ions noted above (e.g., manganese ions, potassium ions, iodine ions) to be adsorbed on a cation exchange resin, thereby finishing the step of allowing As alone to be eluted from the cation exchange resin for removing As.

This step is important. Where the particular removal step is not included, manganese, potassium and iodine having a concentration about 1,000 to 10,000 times as high as the As concentration scatter As, which is the impurity to be measured, so as to make it impossible to introduce As into a mass spectrometric apparatus, resulting in failure to subject As of a low concentration to a quantitative analysis accurately. Such being the situation, it is necessary to remove manganese, potassium and iodine by using an ion exchange resin.

It is desirable for the ion exchange resin to be a cation exchange resin having a styrene-divinyl benzene copolymer as a base material and a sulfone group (—SO₃H) as an exchange group. Also, the particle diameter of the ion exchange resin used is determined appropriately depending on the conditions such as the amount of As subjected to the analysis, the amount of the aqueous solution to be processed, the refining efficiency and the flow rate of the liquid material. In general, it is desirable for the particle diameter of the ion exchange resin to be about 50 to 400 mesh. If the particle diameter noted above is larger than 50 mesh, the flow rate is rendered excessively high, with the result that K and Mn are also caused to pass through the particles of the ion exchange resin. On the other hand, if the particle diameter is smaller than 400 mesh, the flow rate is rendered excessively low, with the result that the total analyzing time is rendered excessively long. It is more desirable for the particle diameter of the ion exchange resin used to fall within a range of between 100 and 200 mesh.

Any of a batch method and a column method can be employed for bringing the solution into contact with the ion exchange resin. However, it is desirable to employ the column method because it is possible for As not to be separated completely from manganese and potassium in the case of employing the batch method. Any material can be used as the material of the batch and the column as far as the material is capable of withstanding the reagents such as hydrofluoric acid and nitric acid, though it is desirable not to use a chlorine-based resin, e.g., polyvinyl chloride, because it is possible for chlorine to be eluted so as to affect the measurement. It is possible to use the batch and the column having an optional inner diameter determined in accordance with the amount of the resin used. Also, the chromatograph using a vibrator and a liquid supply pump can be employed for automation of the operation in each of the batch method and the column method.

(Measurement of As Surface Concentration in Si Semiconductor Wafer Sample)

Finally, performed is an analyzing step for analyzing the target impurity to be analyzed (As), which is contained in the separated solution after the removal step, in, for example, an inductively coupled plasma mass spectrometer. The As dose in the Si semiconductor wafer is obtained by dividing the amount of As, which is obtained, by the area of the Si semiconductor wafer sample, which is obtained in the sample pre-treating step.

The second step in process 3 shown in FIG. 1 will now be described.

(Second Step)

In the second step, the impurity profile in the depth direction of a semiconductor wafer is measured by a plurality of analytical methods owned by a surface analyzing apparatus. It is desirable for the second step to be carried out simultaneously with the first step described previously.

The surface analyzing apparatus used in the present invention is not particularly limited as far as the apparatus permits obtaining the impurity profile in the depth direction. However, it is desirable to use a secondary ion mass spectrometric apparatus or a sputtered neutral mass spectrometric apparatus. In this case, an example of obtaining the As profile in the depth direction of a Si semiconductor wafer will be taken up as a method of obtaining the impurity profile in the depth direction of a semiconductor wafer by the secondary ion mass spectrometry.

Cesium ion (Cs⁺) is used as the primary ion. It is desirable for the energy of the primary ion to be not higher than 1 keV. The resolution in the depth direction can be increased by setting the energy of the primary ion at a level not higher than 1 keV. It is more desirable for the energy of the primary ion to be not higher than 0.8 keV. It is possible to use any type of a mass separator as the analyzing mechanism which analyzes the secondary ions as far as the mass separator is capable of performing the mass separation of the secondary ions and is also capable of detecting the amount of the mass-separated secondary ions. However, it is desirable to use a double focusing mass spectrometer or a quadrupole mass spectroscopy as the mass separator. Also, the As profile can be measured with a high precision, if Cs⁺ and AsSi⁻ are used as the primary ion and the secondary ion, respectively.

In using the secondary ion mass spectrometric apparatus, it is possible to obtain the As profile in the depth direction by, for example, the two methods given below:

1) A method of obtaining the As profile in the depth direction by using Cs primary ions (Cs⁺) having a low energy not higher than 1 keV, the incident angle of the Cs primary ions, i.e., the angle between the passageway of the Cs primary ions and the line normal to the sample surface, being 60°. The secondary ions of AsSi⁻ and Si₂⁻ are measured, and the As profile in the depth direction is obtained from the distribution of the AsSi⁻/Si₂⁻ ion intensity ratio at each depth (analytical point at each depth), in which the AsSi⁻/Si₂⁻ ion intensity ratio is plotted on the ordinate and the measuring time is plotted on the abscissa.

2) A method of obtaining the As profile in the depth direction by using Cs primary ions (Cs⁺) having a low energy not higher than 1 keV, the incident angle of the Cs primary ions, i.e., the angle between the passageway of the Cs primary ions and the line normal to the sample surface, being 60°. The As profile in the depth direction is obtained from the profile of the average Si₂⁻ ion intensity ratio (AsSi⁻/Average Si₂⁻) in the portion where the Si₂⁻ ion intensity is rendered constant, in which the (AsSi⁻/Average Si₂⁻) ion intensity ratio is plotted on the ordinate, and the measuring time is plotted on the abscissa.

The change in the ion intensity ratio obtained by methods 1) and 2) given above in respect of the target sample to be measured was converted into a change in the concentration by measuring a reference sample whose As concentration in the Si semiconductor wafer was known and by comparing the measured value with the As profile of the target sample. Incidentally, in order to suppress the uncertainty effect of the profile in the vicinity of the reference sample (not deeper than 5 nm), it is desirable to use a reference sample having a constant As concentration (impurity concentration) in the depth direction or an As-implanted reference sample having a peak of implantation in a portion deeper than 10 nm. Also, in order to correct the depth of the profile, which was obtained by methods 1) and 2) given above in respect of the target sample, the depth of the crater made by the secondary ion mass spectrometry was measured by, for example, a depth meter. Where the depth of the crater is small (not larger than 100 nm), the error of the depth meter is large. Therefore, it is desirable to correct the depth by the comparison with a sample in which a delta dope is applied to the known depth.

It is possible to obtain two profiles in the depth direction (the concentration on the ordinate, and the corrected depth on the abscissa) based on methods 1) and 2) of the secondary ion mass spectrometry by using the particular means.

The third step in process 4 shown in FIG. 1 will now be described.

(Third Step)

An surface integral is applied to each of the two kinds of the As profiles in the depth direction of the Si semiconductor wafer obtained in the second step so as to obtain two impurity doses based on methods 1) and 2) of the secondary ion mass spectrometry.

Part 1 of fourth step in process 5 shown in FIG. 1 will now be described.

(Part 1 of Fourth Step)

The impurity dose in a semiconductor wafer obtained by the chemical analysis in the first step is represented by “a”. On the other hand, a plurality of (two) impurity doses based on methods 1) and 2) of the secondary ion mass spectrometry in the third step are represented by “b1” and “b2”.

In this step, the plural (two) impurity doses “b1” and “b2” obtained on the basis of methods 1) and 2) of the secondary ion mass spectrometry are compared with the impurity dose “a” in the semiconductor wafer obtained by the chemical analysis that is considered to represent the true dose so as to select the impurity dose closer to the impurity dose “a” from the impurity doses “b1” and “b2”. In this case, it is assumed that the impurity dose “b2” is closer to the impurity dose “a”.

Part 2 of the fourth step in process 6 shown in FIG. 1 will now be described.

(Part 2 of Fourth Step)

Then, it is determined whether or not the ratio of the difference between “a” and “b2” represented by formula (I) given below falls within a range from −0.1 to 0.1, preferably within a range from −0.05 to 0.05:

(a−b2)/a (I)

If the ratio of the difference noted above falls within a range from −0.1 to 0.1, preferably within a range from −0.05 to 0.05, method 2) of the secondary ion mass spectrometry by which the impurity dose “b2” was obtained is the method that permits measuring the true profile in the depth direction.

Also, if the ratio of the difference between “a” and “b2” is smaller than −0.1 or larger than 0.1, each of methods 1) and 2) by which the impurity doses “b1” and “b2” were obtained is the method that is incapable of measuring the true profile in the depth direction. In this case, it is necessary to carry out again the second step by studying and devising another measuring method of the profile in the depth direction.

The description given above covers the case where the dose of the impurity in the semiconductor wafer is measured by the chemical analysis in the first step. It should be noted in this connection that the reference dose is measured by any of the chemical analysis, the nuclear reaction analysis (NRA), the Rutherford backscattering spectrometry (RBS) and the particle induced X-ray emission analysis (PIXE), which permits the measurement of the highest precision. Which of these four methods permits the measurement of the highest precision differs depending on the kind of the impurity. Part 2 of the first step, in which the reference dose is obtained by the nuclear reaction analysis (NRA), and part 3 of the first step, in which reference dose is obtained by the Rutherford backscattering spectrometry (RBS) will now be described.

(Part 2 of First Step)

The method for obtaining the impurity dose in a semiconductor wafer by the nuclear reaction analysis (NRA) will now be described, with the case of obtaining the B dose in a Si semiconductor wafer taken up as an example.

Specifically, a semiconductor wafer sample cut out in the sample pre-treating step is introduced into a nuclear reaction analytical apparatus, followed by evacuating the nuclear reaction analytical apparatus having the sample put therein to set up a vacuum condition. If the semiconductor wafer sample is irradiated with a proton beam accelerated to have an energy of 0.65 MeV, some of B atoms (B atoms having a mass number 11) present in a region within a depth of about 0.3 μm from the surface of the semiconductor wafer bring about a nuclear reaction so as to emit an α particle.

The emitted α particle is detected by an a particle detector (semiconductor detector). If the irradiated p beam is scattered backward by the matrix of the semiconductor wafer so as to enter the α particle detector, the irradiated p beam is a background of the α particle detection generated by the nuclear reaction. Such being the situation, it is desirable to arrange, for example, a Mylar film on the entire surface of the α particle detector so as to prevent the protons that are scattered backward from entering the α particle detector. Also, in order to improve the detection efficiency of the α particle, it is desirable to use an α particle detector as large as possible. It is also desirable to arrange the detector in α position as close to the sample as possible in order to widen the solid angle of the detector.

It is possible to know the B dose in a Si semiconductor wafer used as a target sample by comparing the analytical result (α particle yield) obtained by the nuclear reaction analysis of the target sample with the analytical result (α particle yield) of a Si semiconductor wafer having a known B dose. In this case, it is desirable to make the nuclear reaction analytical conditions of the target sample as close to those of the Si semiconductor wafer having a known B dose as possible. Incidentally, the nuclear reaction analytical conditions include, for example, the amount of charge of the p beam, and the distance between the sample and the α particle detector. Also, where the amount of charge of the p beam differs, the B dose is calculated in view of the difference between p beams. The method described above is for calculating the dose of the isotope element ¹¹B. If another isotope ¹⁰B is contained in the target sample, and if the ratio of presence of the isotope is equal to that of the natural isotope, the B dose is calculated in view of the isotope ratio.

(Part 3 of First Step)

The method of obtaining the impurity dose in a semiconductor wafer by the Rutherford backscattering spectrometry (RBS) will now be described, with the case of obtaining the Sb dose in a Si semiconductor wafer taken up as an example.

Specifically, a semiconductor wafer sample cut out in the sample pre-treating step is put in a Rutherford backscattering spectrometer, followed by evacuating the Rutherford backscattering spectrometer having the sample put therein to set up a vacuum condition. If the semiconductor wafer sample is irradiated with an He ion beam accelerated to have an energy of 2 MeV, some of the incident He ions are scattered backward by the elastic scattering collision with the Si atoms and the Sb atoms present in a region having a depth of about 1 μm from the surface of the semiconductor wafer. The He ions that were scattered backward have an energy dependent on the target atom (Si and Sb atoms in this case). Also, when the He beam passes through the Si semiconductor wafer, the He beam performs the mutual function with the electron within the Si semiconductor wafer because of the non-elastic scattering effect, with the result that the He beam gradually loses the energy in accordance with the depth through which the He beam passes. As a result, it is possible to evaluate the target element profile in the depth direction within the Se semiconductor wafer by analyzing the amount and energy of the He ions that were scattered backward. In this case, it is possible to calculate the Sb dose within the Si semiconductor wafer by comparing the amount of the He ions that were scattered backward by the Sb atoms with the amount of the He ions that were scattered backward by the Si atoms. Incidentally, it is certainly possible to evaluate the impurity profile in the depth direction by the Rutherford backscattering spectrometry. However, the resolution in the depth direction is at most about 10 nm. Such being the situation, it is desirable to employ the secondary ion mass spectrometry or the sputtered neutral mass spectrometry for measuring the distribution of the concentration in the depth direction in the second step.

Example of the present invention will now be described in detail.

### EXAMPLE 1

(Sample Preparation Step)

Prepared in Example 1 was a sample obtained by implanting As ions into a surface region of a Si semiconductor wafer under an energy of 0.5 keV and at dose of 6.0E13 cm⁻². The Si wafer having the As ions implanted thereinto was cut into small pieces each sized at 1 cm×1 cm by using a dicing saw so as to obtain two adjacent samples each sized at 1 cm×1 cm. The size of each sample was measured with a slide gage. Each sample was found to have a vertical length of 1.00 cm and a lateral length of 1.00 cm and, thus, each sample had a surface area of 1.00 cm². Then, in order to remove the cutting dust, the two samples were washed by the ultrasonic cleaning in a pure water. One of these samples, i.e., sample A, was used in the first step, and the other sample, i.e., sample B, was used in the second step.

(First Step)

The As concentration in the Si semiconductor wafer was measured by a chemical analysis. The reason for employing the chemical analysis was as follows. Specifically, in order to analyze the As concentration in a semiconductor wafer, e.g., a Si semiconductor wafer, by employing the nuclear reaction analysis (NRA) having a high reaction cross sectional area, it is necessary to set the energy of the ion beam used for bringing about a nuclear reaction at a level higher than the ordinary level not higher than 3 MeV, making it difficult to use the general purpose nuclear reaction analytical apparatus. Incidentally, the nuclear reaction analysis can be employed without difficulty, if a nuclear reaction analytical apparatus capable of generating a high energy ion beam is available. Also, if the Rutherford backscattering spectrometry is employed in the case where the As dose in the semiconductor wafer, e.g., a Si semiconductor wafer, is not higher than the order of about E13 cm⁻², the sensitivity tends to be rendered insufficient. Of course, the Rutherford backscattering spectrometry can be employed without difficulty in the case of analyzing the As concentration not lower than about E15 cm⁻². Under the circumstances, the chemical analysis was employed in Example 1 as the method for analyzing the As concentration with a high precision and with a high accuracy.

<First Reaction Step>

Sample A was put in a bottle having an inner volume of 250 ml and made of a fluorocarbon polymers. Then, 10 ml of a potassium permanganate solution (6% w/v), 7 ml of a high purity nitric acid, and 7 ml of a high purity hydrofluoric acid were successively poured into the bottle, and the bottle was put on a hot plate heated to 200° C. so as to heat the mixed solution and decompose sample A. In decomposing the sample, a lid was put lightly on the bottle so as to prevent the potassium permanganate solution from being scattered, and the lid was removed after the reaction was stabilized. Also, the potassium permanganate solution was added as required when the color (pale pink) of the potassium permanganate solution was eliminated by the reaction between potassium permanganate and nitric acid.

After the reaction mixture was cooled, a high purity hydrogen peroxide solution was added to the reaction mixture several times in an amount of 0.1 ml to 0.5 ml each time until the color (pale pink) of the potassium permanganate solution was eliminated to reduce potassium permanganate. Then, the reaction mixture was poured into a beaker having an inner volume of 100 ml and made of a fluorocarbon polymers, followed by putting the beaker on a hot plate heated to 230° C. so as to evaporate the volatile components and, thus, to condense the reaction mixture.

<Second Reaction Step>

After the condensed reaction mixture was cooled, 0.1 ml of a high purity nitric acid and 0.5 ml of a high purity hydrogen peroxide aqueous solution were poured into the beaker so as to dissolve the manganese condensate formed in the step of evaporating the volatile components for condensing the reaction mixture. Then, the reaction mixture was poured into a graduated flask having an inner volume of 50 ml and made of a fluorocarbon polymers, followed by adding water to the reaction mixture to permit the volume of the reaction mixture to be increased to reach the gage mark of the graduated flask.

<Cation Exchange>

A strongly acidic divinyl benzene gel having an average particle diameter of 75 to 150 mesh, which was used as the cation exchange resin, was loaded in an amount of 10 g in a polypropylene column having a length of 150 mm and a diameter of 5 mm, a polypropylene wool being loaded in the bottom of the polypropylene column, followed by sufficiently washing and swelling the cation exchange resin with a pure water and nitric acid. Then, 1 ml of the processed liquid obtained in the second reaction step was dispensed by an Eppendorf pipette and allowed to flow into the cation exchange column. The particular operation was repeated 10 times so as to allow 10 ml in total of the process solution to flow through the cation exchange column. Further, 5 ml of water was allowed to flow through the cation exchange column three times so as to allow As to elute into 25 ml in total of the liquid material.

Then, all the eluate was recovered in a container having an inner volume of 30 ml and made of a fluorocarbon polymers, and the amount of As in the solution was measured by an inductively coupled plasma mass spectrometric apparatus, with the result that the amount of As was found to be 7.85 ng. Since the area of sample A was 1.00 cm², the dose using the number of atoms was 6.30E13 atoms/cm². The dose thus obtained was used as the reference dose.

(Second Step)

In this step, the profile of the As concentration in the depth direction of sample B prepared in the sample pre-treating step was measured by using the quadrupole secondary ion mass spectroscopy.

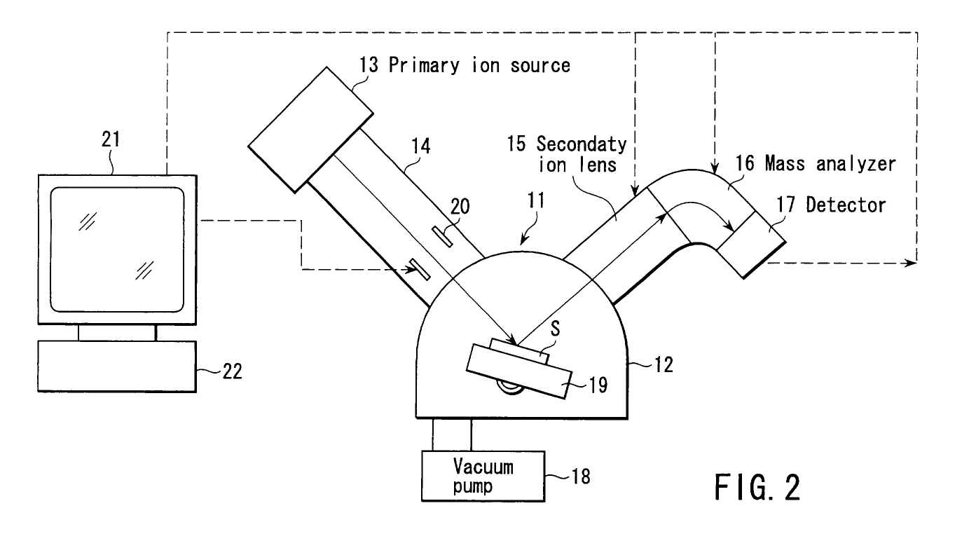

First of all, the secondary ion mass spectrometric apparatus used in this step will now be described with reference to FIG. 2. Specifically, FIG. 2 schematically shows the construction of the secondary ion mass spectrometric apparatus 11 used in Example 1.

As shown in FIG. 2, the secondary ion mass spectrometric apparatus 11 comprises a sample chamber 12 in which a sample S is arranged, a primary ion source 13 for generating a primary ion beam for sputtering the surface of the sample S, a primary ion gun column 14 for converging the primary ions generated from the primary ion source 13 and for forming a raster of the primary ions, a secondary ion lens 15 for withdrawing the secondary ions generated from the surface of the sample S housed in the sample chamber 12, a mass analyzer used as a mechanism which analyzes the secondary ions sent from the secondary ion lens 15, a detector 17 used as a mechanism which detects the secondary ions subjected to the mass separation, a vacuum pump 18 for maintaining the vacuum state within the surface analytical apparatus, and a sample manipulators 19 having the sample S disposed thereon and serving to adjust the angle and position for irradiating the sample S with the primary ion beam. Also, an electrode 20 for forming a raster of the primary ions within the primary ion gun column 14 is arranged within the primary ion gun column 14. Further, a computer 21 which controls the electrode 20, the secondary ion lens 15 and the mass analyzer 16 is electrically connected to the electrode 20, the secondary ion lens 15 and the mass analyzer 16. Still further, the signal detected by the detector 17 can be transmitted to the computer 21 that is electrically connected to the detector 17. Incidentally, the operation of the computer 21, the input of the data, etc. are carried out on a keyboard 22 electrically connected to the computer 21.

The method of measuring the impurity profile in the depth direction of the sample, which is carried out by using the secondary ion mass spectrometric apparatus described above, will now be described.

(1) In the first step, the sample S is introduced into the sample chamber 12.

(2) The operation of the primary ion source 13 is started up.

(3) The primary ion gun column 14 is operated so as to adjust the primary ion beam to have a prescribed energy, a prescribed current amount, and a prescribed beam diameter.

(4) The sample manipulator 19 having the sample S disposed thereon is adjusted so as to adjust the incident angle of the primary ion beam on the sample S at a prescribed value and to set the irradiating position of the primary ion beam at a prescribed position.

(5) The secondary ion lens 15 and the mass analyzer 16 are adjusted in order to guide efficiently the secondary ions generated on the surface of the sample S into the detector 17 and in order to obtain a prescribed mass resolution.

(6) The keyboard 22 of the computer 21 is operated so as to transmit the signal for starting the measurement, which is generated from the computer 21, to the primary ion electrode 20, the secondary ion lens 15 and the mass analyzer 16. As a result, the sputtering of the sample S is started at a prescribed raster width so as to carry out the prescribed mass separation of the secondary ion species and the detection of the secondary ion intensity. Then, a signal denoting the intensity of the prescribed secondary ions detected by the detector 17 is transmitted to the computer 21. Further, the profile of the prescribed secondary ions in the depth direction (the secondary ion intensity ratio being plotted on the ordinate, and the measuring time being plotted on the abscissa) is obtained by continuously carrying out the sputtering and by allowing the computer to take in continuously the signal of the prescribed secondary ions.

(7) In order to correct the depth of the profile in the depth direction, the depth of the crater made by the secondary ion mass spectrometry is measured by, for example, a depth meter. Then, the time on the abscissa of the graph denoting the profile in the depth direction is converted into the depth by supplying the depth obtained by the depth meter into the computer 21 by operating the keyboard 22.

(8) In respect of a reference sample having a known impurity concentration, the profile of the secondary ion concentration in the depth direction (the secondary ion intensity ratio being plotted on the ordinate, and the measuring time being plotted on the abscissa) is obtained as described previously under item (6) described above and, then, a prescribed relative sensitivity factor of the secondary ion is calculated from the obtained profile. The secondary ion intensity ratio plotted on the ordinate of the graph showing the profile in the depth direction is converted into a concentration (atoms/cm³) by supplying the calculated relative sensitivity factor into the computer 21 through the keyboard 22 so as to obtain the profile in the depth direction in respect of the aimed impurity contained in the sample. The obtained data on the profile in the depth direction is stored in the computer 21.

The As profile in the depth direction of the sample B was obtained by using the secondary ion mass spectrometric apparatus described above under the three measuring conditions given below. Incidentally, a reference sample having an As implantation energy of 20 keV and a dose of 1E15 cm⁻² was prepared as a reference sample having a known As concentration, which was used in converting the profile of ion intensity ratio in the depth direction into the profile of the concentration in the depth direction.

1) Measured were the AsSi⁻ secondary ion concentration and the Si₂⁻ secondary ion concentration by using Cs primary ions (Cs⁺) having an energy of 0.6 keV, the incident angle of the Cs primary ion beam, i.e., the angle between the passageway of the Cs primary ion beam and the line normal to the sample surface, being 60°. Then, the As concentration profile in the depth direction was obtained from the AsSi⁻/Si₂⁻ ion intensity ratio at each depth (analytical point at each depth).

2) Measured were the AsSi⁻ secondary ion concentration and the Si⁻ secondary ion concentration by using Cs primary ions (Cs⁺) having an energy of 0.6 keV, the incident angle of the Cs primary ion beam, i.e., the angle between the passageway of the Cs primary ion beam and the line normal to the sample surface, being 60°. Then, the As concentration profile in the depth direction was obtained from the AsSi⁻/Si⁻ ion intensity ratio at each depth (analytical point at each depth).

3) Measured were the AsSi⁻ secondary ion concentration and the Si₂⁻ secondary ion concentration by using Cs primary ions (Cs⁺) having an energy of 0.6 keV, the incident angle of the Cs primary ion beam, i.e., the angle between the passageway of the Cs primary ion beam and the line normal to the sample surface, being 60°. Then, the As concentration profile in the depth direction was obtained from the ratio of the average Si₂⁻ ion intensity (AsSi⁻/Average Si₂⁻) at a deep portion (5 nm to 30 nm) in which the Si₂⁻ ion intensity is rendered constant.

In each of the three measuring conditions described above, used were the Cs primary ion having an energy of 0.6 keV and the AsSi⁻ secondary ion because the particular combination permits measuring the As profile with the highest sensitivity. The energy of the primary ion was set at 0.6 keV in order to improve the resolution in the depth direction. If the energy of the primary ion is not higher than 0.8 keV, it is possible to perform the measurement with a high resolution in the depth direction.

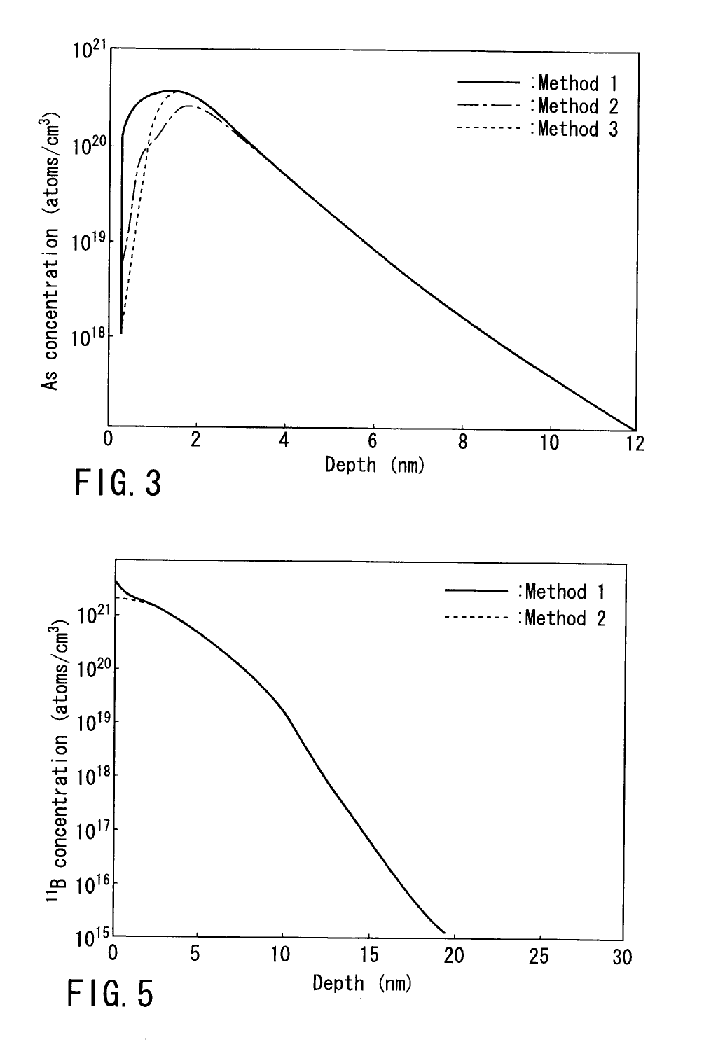

FIG. 3 is a graph showing the As concentration profile in the depth direction of the sample B, which were obtained under the measuring conditions 1) to 3) given above. In the graph of FIG. 3, the depth (nm) of the sample B is plotted on the abscissa, with the As concentration being plotted on the ordinate.

As apparent from FIG. 3, the profiles of the As concentration in the depth direction, which were measured under the three measuring conditions 1), 2), 3) given above of the secondary ion mass spectrometry, are rendered equal to each other in the region deeper than 3 nm from the surface, but are rendered different from each other in the region not deeper than 3 nm from the surface. In other words, it is supported that the profiles of the concentration in the depth direction obtained under the measuring conditions 1), 2), 3) are not equal to each other.

(Third Step)

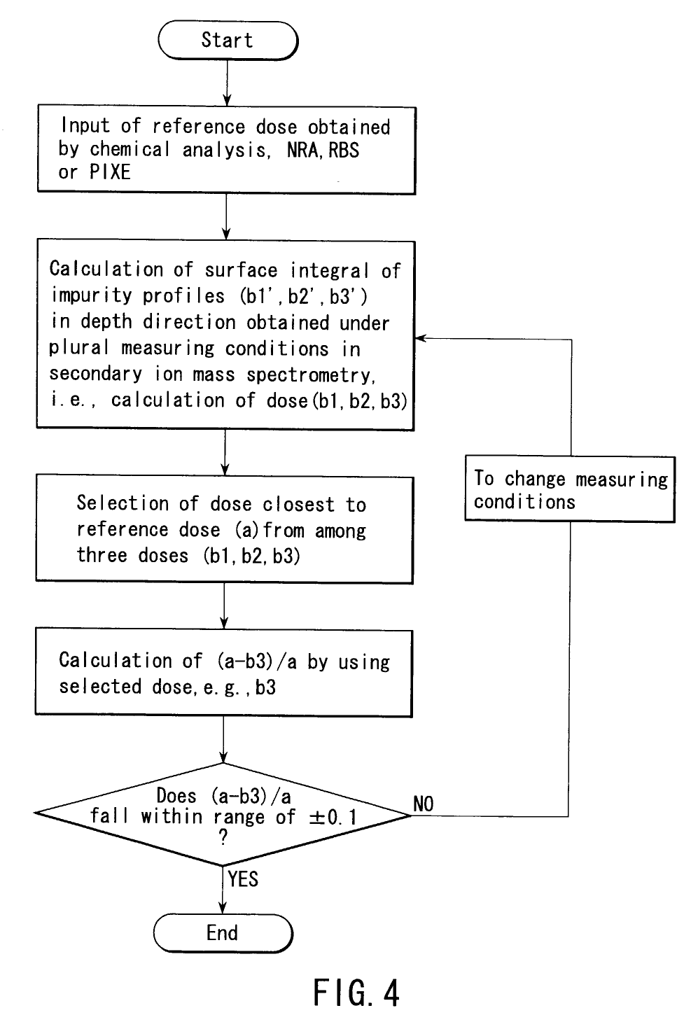

In the first step, the reference dose (a) obtained in the first step was supplied to the computer 21, as shown in the flow chart given in FIG. 4. Then, the computer 21 was caused to calculate the doses b1, b2 and b3, i.e., the surface integral for each of the profiles (b1″ b2′, b3′) of the As concentration in the depth direction of the sample B (Si semiconductor wafer). Those profiles are stored in the computer 21 in the second step.

Table 1 shows the obtained three As doses b1, b2, b3 based on the measuring conditions 1), 2), 3) of the secondary ion mass spectrometry. Incidentally, Table 1 also shows the As dose (a) in the sample A (semiconductor wafer) obtained by the chemical analysis in the first step.

TABLE 1
As dose in sample B obtained under measuring
conditions (1), (2), (3) of secondary ion
mass spectrometry

| | Measuring | Measuring | Measuring | |
|---|---|---|---|---|
| | condition | condition | condition | Chemical |
| | (1) | (2) | (3) | analysis |
| | b1 | b2 | b3 | a |
| As dose | 8.8E13 | 5.6E13 | 6.8E13 | 6.3E13 |
| (atoms/cm²) | | | | |

(Fourth Step)

The As dose (a), 6.3E13 atoms/cm², in a semiconductor wafer obtained by the chemical analysis in the first step was compared with the As dose shown in Table 1, which were obtained under the measuring conditions 1), 2), 3) of the secondary ion mass spectrometry in the third step, by the computer 21, and the computer 21 was caused to select the result obtained under the measuring condition 3), which was closest to the result obtained by the chemical analysis and representing the true value of the As dose. Also, the computer 21 was caused to calculate the value of {(a−b3)/a}, where “a” represents the reference dose obtained by the chemical analysis, and “b3” represents the dose obtained under the measuring condition 3) of the secondary ion mass spectrometry. The calculated value of {(a−b3)/a} was found to be +0.08 falling within a range from −0.1 to +0.1. In conclusion, it has been found that the measuring condition 3) of the secondary ion mass spectrometry was the measuring condition that permits measuring the profile of the concentration in the depth direction, said measured profile representing the true profile or the profile close to the true profile.

If the value of {(a−b3)a} noted above should be smaller than −0.1 or larger than +0.1, the operation is brought back to the screen for the measurement of the secondary ion mass spectrometry so as to cause the computer 21 to measure the profile of the As concentration in the depth direction of the sample B by changing the measuring conditions of the secondary ion mass spectrometry.

The change in the measuring conditions of the secondary ion mass spectrometry includes, for example, the lowering of the energy of the Cs primary ion (Cs⁺), e.g., the lowering to 0.4 keV, in the measuring conditions 1), 2), 3) given above. If the energy of the primary ion is lowered, it is possible to perform the analysis more accurately.

After the secondary ion mass analysis performed under the changed measuring conditions, the dose is calculated by the computer 21 from the surface integral of the profile of the concentration in the depth direction, and the calculated dose is compared with the reference dose obtained in the first step described previously.

Further, the profile of the As concentration in the depth direction of a Si semiconductor wafer, which was subjected to a plurality of manufacturing steps in the research and development of the Si semiconductor device such as a plurality of ion implantation steps and a plurality of heat treating steps, is measured under the measuring condition 3) which permits obtaining the true profile of the As concentration in the depth direction. Since the plural profiles of the As concentration in the depth direction thus obtained represents the true As profile, it is possible to determine accurately which Si semiconductor manufacturing process is most excellent by comparing and studying the shape of the profile, the As dose obtained therefrom, etc. In other words, since it is possible to compare and study accurately a plurality of semiconductor manufacturing processes in the stage of the research and development, it is possible to carry out the research and development efficiently.

Also, the profile of the As concentration in the depth direction in the Si semiconductor factory is measured by using the measuring condition 3) that permits obtaining the true profile of the As concentration. It is possible to perform accurately the defective analysis of the Si semiconductor device derived from the abnormality in the shape of the profile of the As concentration and the dose by comparing and studying the shapes of the profile of the As concentration in the depth direction of each of a good sample and a defective sample and by comparing and studying the As doses derived from those profiles.

### COMPARATIVE EXAMPLE 1

The operation was performed as in Example 1, except that the first step was not carried out. As shown in Table 1 given previously, the doses obtained under the measuring conditions 1), 2), 3) of the secondary ion mass spectrometry differ from each other. Therefore, it is unclear which value represents the true value or the value close to the true value in the case where the secondary ion mass spectrometry alone was carried out. In other words, it is supported that it is impossible to determine the true profile of the As concentration in the depth direction in Comparative Example 1.

It should also be noted that, since the true profile of the As concentration in the depth direction cannot be obtained, the research efficiency and the defect analyzing efficiency are deteriorated in the research and development of a Si semiconductor device and in the Si semiconductor device factory.

### EXAMPLE 2

(Sample Preparation Step)

In Example 2, prepared was a sample obtained by introducing ¹¹B into a surface region of a Si semiconductor wafer by the ion implantation under an accelerating energy of 0.5 keV and at a dose of 1.0E15 cm⁻². The Si semiconductor wafer having B ions implanted thereinto was cut into pieces each sized at about 1 cm×1 cm by using a diamond cutter so as to obtain two adjacent samples each sized at about 1 cm×1 cm. Then, a nitrogen gas was blown against the two samples in order to remove the cutting dust. One of these samples (sample C) was used in the first step, and the other sample (sample D) was used in the second step.

(First Step)

The analysis of B in the Si semiconductor wafer (sample C) was performed by the nuclear reaction analysis. It should be noted in this connection that, if the B analysis is carried out by the chemical analysis described previously in conjunction with Example 1, it is possible for B to be evaporated during the process, resulting in failure to recover the entire amount of B. Also, since the B dose in the Si semiconductor wafer is not higher than about E15 cm⁻², the sensitivity is too low to carry out the B analysis by the Rutherford backscattering spectrometry. Such being situation, the nuclear reaction analysis was selected in Example 2 as the method that permits the B analysis with a high precision and with a high accuracy.

In the first step, sample C was introduced into a nuclear reaction analyzing apparatus, followed by evacuating the nuclear reaction analyzing apparatus having the sample C introduced therein so as to set up a vacuum environment. When the semiconductor wafer sample was irradiated with a proton beam accelerated to have an energy of 0.65 MeV, a nuclear reaction was brought about in some of the ¹¹B atoms introduced in the semiconductor wafer by the ion implantation, with the result that α particles were released to the outside of the sample. The released α particles were detected by a semiconductor detector, and the α particles thus detected were counted until the charge amount of the sample current caused by the proton beam irradiation was changed to 100 μC (micro coulombs), with the result that the number of counts was 3232 counts.

Incidentally, the entire surface of the detector was covered with a Mylar film so as to prevent the protons scattered backward from entering the detector. Also, in order to improve the detecting efficiency of the α particles, used was a detector having a sensitive area of 2000 mm², and the distance between the sample and the detector was set at 10 cm so as to ensure a large solid angle.

On the other hand, prepared was a sample (reference sample) obtained by implanting ¹¹B into a surface region of a Si semiconductor wafer by the ion implantation under an accelerating energy of 5 keV and at a dose of 1.0E16 cm⁻². If the accelerating energy for the ion implantation is set at 5 keV, it is possible to introduce ¹¹B into the Si semiconductor wafer in a desired dose. In other words, the dose of ¹¹B in the reference sample was 1.0E16 cm⁻², which is equal to the dose. The B analysis was carried out in respect of the reference sample noted above by the nuclear reaction analysis under the conditions equal to those described previously, with the result that the number of counts of the α particles was 29381 counts.

Since the target sample to be measured (sample C) and the reference sample were measured under the same conditions including the charge amount of the proton, the dose of ¹¹B of the target sample (sample C) can be obtained by the obtained ratio of the number of counts of the α particles, i.e., the ratio A/B, where A represents the number of counts of the α particles in the target sample (sample C), and B represents the number of counts of the α particles in the reference sample, and by the dose of the reference sample. The dose of ¹¹B in the target sample (sample C), which was obtained in this manner, was found to be 1.10E15 atoms/cm². This dose is used as the reference dose.

(Second Step)

In this step, the profile of the ¹¹B concentration in the depth direction of the semiconductor wafer sample (sample D) prepared in the sample preparation step was measured by the quadrupole secondary ion mass spectroscopy.

The profile of the ¹¹B concentration in the depth direction of sample D was obtained under the two conditions given below by using the secondary ion mass spectrometric apparatus shown in FIG. 2. Incidentally, a Si semiconductor wafer having a ¹¹B concentration of 6E18 cm⁻³, which was uniform in the depth direction, was prepared as a reference sample having a known B concentration, which is used in converting the profile of the secondary ions in the depth direction into the profile of the concentration in the depth direction.

1) B⁺ ion was measured by using the oxygen primary ion (O₂⁺) having an energy of 0.5 keV, the incident angle of the primary oxygen ion beam being 0°, so as to obtain the profile of B in the depth direction from the profile of the B⁺ secondary ions in the depth direction.

2) B⁺ secondary ion was measured by using the oxygen primary ion (O₂⁺) having an energy of 0.5 keV, the incident angle of the oxygen primary ion beam being 45°, while blowing an oxygen gas against the sample surface, so as to obtain the profile of B in the depth direction from the profile of the B⁺ secondary ions in the depth direction. The obtained data on the profile of the concentration in the depth direction is stored in the computer.

In each of the measuring conditions 1) and 2) given above, the oxygen primary ion (O₂⁺) having an energy of 0.5 keV was used as the primary ion, and the B⁺ ion was used as the secondary ion, because the particular combination permits measuring B with the highest sensitivity. It should be noted that a high resolution in the depth direction can be obtained by setting the energy of the primary ion at a level not higher than 0.5 keV.

FIG. 5 is a graph showing the profiles of the ¹¹B distribution in the depth direction of the sample D, which were obtained under the measuring conditions 1) and 2) given above. In the graph of FIG. 5, the depth (nm) of the sample D is plotted on the abscissa, with the ¹¹B concentration (atoms/cm³) being plotted on the ordinate.

As apparent from FIG. 5, the profiles of the ¹¹B concentration in the depth direction, which were measured under the measuring conditions 1) and 2) of the secondary ion mass spectrometry, were equal to each other in the region deeper than 2 nm from the surface, but differed from each other in the region not deeper than 2 nm from the surface. In other words, FIG. 5 shows that the profiles of the concentration in the depth direction obtained under the measuring conditions 1) and 2) are not equal to each other.

(Third Step)

In the first step, the reference dose (a) obtained in the first step was supplied to the computer. Then, the surface integral for each of the profiles b1′ and b2′ of the B concentration in the depth direction of the sample D (Si semiconductor wafer) stored in the computer in the second step, i.e., the doses b1 and b2, was calculated by the computer.

Table 2 shows the obtained two ¹¹B doses based on the measuring conditions 1) and 2) of the secondary ion mass spectrometry. Incidentally, Table 2 also shows the ¹¹B dose in the sample C (semiconductor wafer) obtained by the nuclear reaction analysis of the first step.

TABLE 2
¹¹B dose in sample D obtained under
measuring conditions (1), (2) of secondary
ion mass spectrometry

| | Measuring | Measuring | Nuclear |
|---|---|---|---|
| | condition | condition | reaction |
| | (1) | (2) | analysis |
| | b1 | b2 | a |
| ¹¹B dose | 1.23E13 | 1.05E13 | 1.10E15 |
| (atoms/cm²) | | | |

(Fourth Step)

The dose (a), i.e., 1.10E15 atoms/cm², of ¹¹B in the semiconductor wafer, which was obtained by the nuclear reaction analysis in the first step, was compared by the computer with the doses of ¹¹B shown in Table 2, which were obtained under the measuring conditions 1) and 2) of the secondary ion mass spectrometry in the third step, so as to cause the computer to select the result obtained under the measuring condition 2), which was closest to the result obtained by the nuclear reaction analysis and representing the true dose. Also, the value of {(a−b2/a}, where “a” represents the dose obtained by the nuclear reaction analysis, and “b2” represents the dose obtained under the measuring condition 2) of the secondary ion mass spectrometry, was found to be −0.05 falling within the range from −0.1 to +0.1. In conclusion, it has been found that the measuring condition 2) of the secondary ion mass spectrometry represents the measuring condition that permits measuring the true profile of the concentration in the depth direction or the profile close to the true profile.

If the value of {(a−b2)/a} is smaller than −0.1 or larger than +0.1, the computer changes the measuring condition of the secondary ion mass spectrometry and is brought back to the screen for the measurement by the secondary ion mass spectrometry so as to measure the profile of the ¹¹B concentration in the depth direction of the sample D.

The change in the measuring conditions of the secondary ion mass spectrometry includes, for example, the lowering the energy of the oxygen primary ion (O₂⁺) in each of the measuring conditions 1) and 2), e.g., the lowering to 0.25 keV. If the energy of the primary ion is lowered, it is possible to perform the analysis more accurately.

After the secondary ion mass spectrometry under the changed measuring conditions, the dose is calculated by the computer from the surface integral of the profile of the concentration in the depth direction, and the calculated dose is compared with the reference dose obtained in the first step described previously.

Then, the profile of the boron concentration in the depth direction of the Si semiconductor wafer after a plurality of manufacturing steps such as a plurality of ion implantation steps and a plurality heat treating steps in the research and development of the Si semiconductor device is measured under the measuring condition 2) that permits obtaining the true profile of the boron concentration in the depth direction. Since the obtained plural profiles of the boron concentration in the depth direction represents the true boron profile, it is possible to determine accurately which manufacturing process of the Si semiconductor device is most excellent by comparing and studying the shape of the profile and the boron dose obtained therefrom. In other words, since the comparison and study of a plurality of manufacturing processes of the Si semiconductor device can be performed accurately in the stage of the research and development, the research and development can be performed efficiently.

Also, the profile of the boron concentration in the depth direction of the Si semiconductor wafer is measured in the Si semiconductor device factory under the measuring condition 2) that permits obtaining the true profile of the boron concentration in the depth direction. It is possible to perform accurately the defective analysis of the Si semiconductor device derived from the abnormality in the shape of the boron profile and the dose by comparing and studying the shapes of the profile of the boron concentration in the depth direction and the boron dose derived therefrom in respect of a good sample and a defective sample.

### COMPARATIVE EXAMPLE 2

The operation was performed as in Example 2, except that the first step was not carried out. As shown in Table 2 given previously, the doses obtained under the measuring conditions 1) and 2) of the secondary ion mass spectrometry differ from each other. Therefore, it is unclear which value represents the true value or the value close to the true value in the case where the secondary ion mass spectrometry alone was carried out. In other words, it is supported that it is impossible to determine the true profile of the concentration in the depth direction in Comparative Example 2.

It should also be noted that, since the true profile of the boron concentration in the depth direction cannot be obtained, the research efficiency and the defect analyzing efficiency are deteriorated in the research and development of a Si semiconductor device and in the Si semiconductor device factory.

### EXAMPLE 3

The reference dose was measured by the chemical analysis in respect of a Si semiconductor wafer containing P (phosphorus) as an impurity. Also, a plurality of profiles of the P concentration in the depth direction of the semiconductor wafer were obtained by using a secondary ion mass spectrometric apparatus similar to that described previously in conjunction with Example 1. In this secondary ion mass spectrometry, Cs⁺ was used as the primary ion and PSi⁻ was used as the secondary ion. Each profile of the concentration in the depth direction was converted into the dose by the method similar to that described previously in conjunction with Example 1, and the dose thus converted was compared with the reference dose. It has been found that the dose obtained under the measuring condition involving the use of (PSi⁻/Average Si₂⁻) as the secondary ion intensity ratio was closest to the reference dose.

Also, the value of {(a−b)/a}, where “a” represents the reference dose obtained by the chemical analysis, and “b” represents the dose obtained under the measuring condition involving the use of (PSi⁻/Average Si₂⁻) as the secondary ion intensity ratio, was found to be +0.03, which falls within the range from −0.1 to +0.1. In other words, it has been found that the measuring condition using (PSi⁻/Average Si₂⁻) as the secondary ion intensity ratio represents the measuring condition that permits obtaining the true profile of the concentration in the depth direction or the profile close to the true profile.

### EXAMPLE 4

(Sample Preparation Step)

In Example 4, prepared was a sample obtained by introducing ¹²¹Sb (antimony) into a surface region of a Si semiconductor wafer by an ion implantation method under an accelerating energy of 1 keV and at a dose of 1.2E14 cm⁻². The Si semiconductor wafer having the Sb ions implanted thereinto was cut into pieces each sized at about 3 mm×3 mm by a diamond cutter so as to obtain two adjacent samples each sized at about 3 mm×3 mm. Then, a nitrogen gas was blown against these two samples so as to remove the cutting dust. One of these samples (sample E) was used in the first step, and the other sample (sample F) was used in the second step.

(First Step)

The particle induced X-ray emission analysis was employed for analyzing Sb in the Si semiconductor wafer (sample E). It is difficult to analyze Sb in the Si semiconductor wafer by the nuclear reaction analysis because Sb is not sensitive to the nuclear reaction. It is also difficult to analyze Sb in the Si semiconductor wafer by the Rutherford backscattering spectrometry because the dose of Sb to be measured was too small. Further, it is difficult to analyze Sb in the Si semiconductor wafer by the chemical analysis because the sample to be analyzed was sized at 3 mm×3 mm, which was too small to be measured by the chemical analysis. Under the circumstances, employed was the particle induced X-ray emission analysis which permits analyzing Sb with a high precision and at a high accuracy.

In the first step, the sample E was introduced into a particle induced X-ray emission analytical apparatus, followed by evacuating the particle induced X-ray emission analytical apparatus so as to set up a vacuum condition within the apparatus. When the semiconductor wafer sample was irradiated with a proton beam accelerated to have an energy of 1 MeV, Sb within the sample emitted a characteristic X-ray because of the mutual function between the irradiating proton beam and the sample. The characteristic X-ray emitted from Sb was detected by an X-ray detector (energy dispersive semiconductor detector) and measured until the amount of charge of the sample current generated by the proton beam irradiation was increased to reach 100 μC (micro coulombs). After the back ground was removed from the resultant X-ray spectrum, the X-ray yield of the characteristic X-ray generated from Sb was added up, with the result that the number of counts was found to be 4498 counts.

On the other hand, prepared was a sample (reference sample) obtained by introducing ¹²¹Sb into a surface region of a Si semiconductor wafer by means of an ion implantation under an accelerating energy of 20 keV and at a dose of 1.0E16 cm⁻². If the accelerating energy in performing the ion implantation is set at 20 keV, Sb can be implanted into the Si semiconductor wafer in a desired dose. In other words, the dose of ¹²¹Sb in the reference sample is rendered equal to the dose, i.e., 1.0E16 cm⁻². The Sb analysis was applied to the reference sample by the particle induced X-ray emission analysis under the conditions equal to those described above, and the X-ray yield of the characteristic X-ray was added up, with the result that the number of counts was found to be 335675 counts.

Since the target sample (sample E) and the reference sample were measured under the same conditions including the amount of charge of the proton beam, the dose of Sb in the target sample (sample E) can be obtained by both of the ratio of the number of counts of the obtained characteristic X-ray integration, which is obtained by dividing the number of counts of the characteristic X-ray in the target sample (sample E) by the number of counts of the characteristic X-ray in the reference sample, and the dose of the reference sample. As a result, the dose of Sb in the target sample (sample E) was found to be 1.34E14 atoms/cm². The dose thus obtained is used as the reference dose.

(Second Step)

In this step, the profile of the ¹²¹Sb concentration in the depth direction of a semiconductor wafer sample (sample F) prepared in the sample preparation step was measured by the quadrupole secondary ion mass spectroscopy.

Specifically, the profiles of the ¹²¹Sb concentration in the depth direction of the sample F were obtained by the three methods given below by using the secondary ion mass spectrometric apparatus shown in FIG. 2. Incidentally, a reference sample into which ¹²¹Sb was introduced by means of an ion implantation under an accelerating energy of 30 keV and at a dose of 1E15 cm⁻² was prepared as a reference sample having a known Sb concentration, said reference sample being used when the profile of the secondary ions in the depth direction was converted into the profile of the concentration in the depth direction.

1) Measured were the SbSi⁻ secondary ion concentration and the Si₂⁻ secondary ion concentration by using Cs primary ions (Cs⁺) having an energy of 0.6 keV, the incident angle of the Cs primary ion beam, i.e., the angle between the passageway of the Cs primary ion beam and the line normal to the sample surface, being 60°. Then, the profile of the Sb concentration in the depth direction was obtained from the SbSi⁻/Si₂⁻ ion intensity ratio at each depth (analytical point at each depth).

2) Measured were the SbSi⁻ secondary ion concentration and the Si⁻ secondary ion concentration by using Cs primary ions (Cs⁺) having an energy of 0.6 keV, the incident angle of the Cs primary ion beam, i.e., the angle between the passageway of the Cs primary ion beam and the line normal to the sample surface, being 60°. Then, the profile of the Sb concentration in the depth direction was obtained from the SbSi⁻/Si⁻ ion intensity ratio at each depth (analytical point at each depth).

3) Measured were the SbSi⁻ secondary ion concentration and the Si₂⁻ secondary ion concentration by using Cs primary ions (Cs⁺) having an energy of 0.6 keV, the incident angle of the Cs primary ion beam, i.e., the angle between the passageway of the Cs primary ion beam and the line normal to the sample surface, being 60°. Then, the profile of the Sb concentration in the depth direction was obtained from the ratio of the average Sb₂⁻ ion intensity (SbSi⁻/Average Si₂⁻) at a deep portion (5 nm to 30 nm) in which the Si₂⁻ ion intensity is rendered constant.

In each of the three measuring conditions described above, used were the Cs primary ion having an energy of 0.6 keV and the SbSi⁻ secondary ion because the particular combination permits measuring the Sb concentration with the highest sensitivity. The energy of the primary ion was set at 0.6 keV in order to improve the resolution in the depth direction. If the energy of the primary ion is not higher than 0.8 keV, it is possible to perform the measurement with a high resolution in the depth direction.

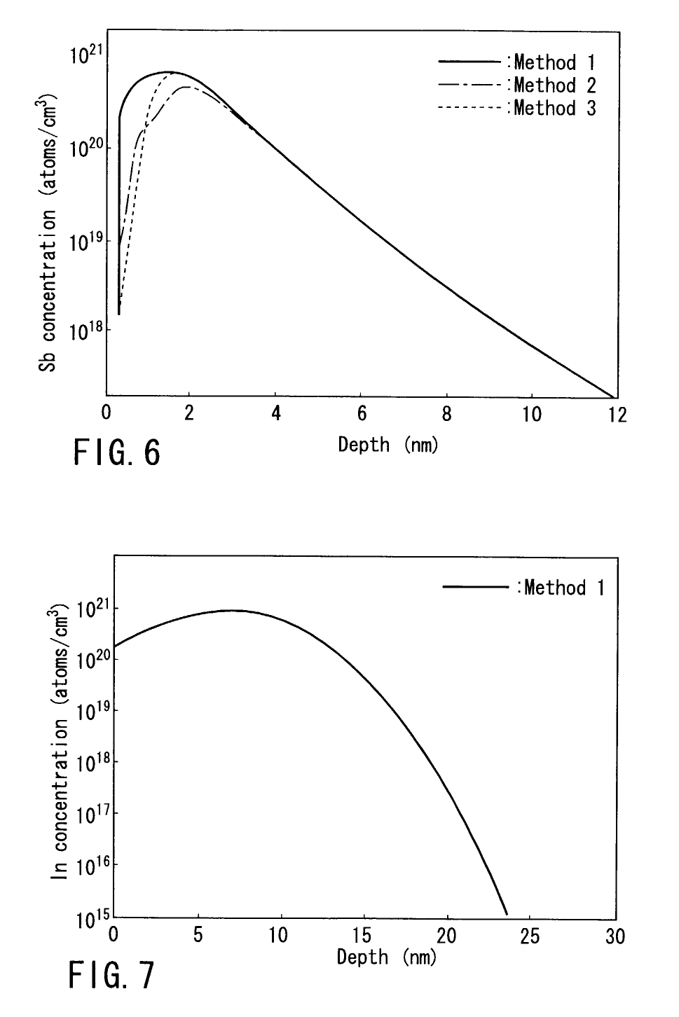

FIG. 6 is a graph showing the profiles of the Sb concentration in the depth direction of the sample F, which were obtained under the measuring conditions 1) to 3) given above. In the graph of FIG. 6, the depth (nm) of the sample F is plotted on the abscissa, with the Sb concentration (atoms/cm³)being plotted on the ordinate.

As apparent from FIG. 6, the profiles of the Sb concentration in the depth direction, which were measured under the three measuring conditions 1), 2), 3) given above of the secondary ion mass spectrometry, are rendered equal to each other in the region deeper than 3 nm from the surface, but are rendered different from each other in the region not deeper than 3 nm from the surface. In other words, it is supported that the profiles of the concentration in the depth direction obtained under the measuring conditions 1), 2), 3) are not equal to each other.

(Third Step)

In the first step, the reference dose (a) obtained in the first step was supplied to the computer 21. Then, the profiles (b1′ b2′, b3′) of the Sb concentration in the depth direction of the sample F (Si semiconductor wafer) in the second step was supplied to the computer 21. The computer 21 was caused to calculate the doses b1, b2 and b3, i.e., the surface integral for each of the profiles (b1′ b2′, b3′).

Table 3 shows the obtained three Sb doses b1, b2, b3 based on the measuring conditions 1), 2), 3) of the secondary ion mass spectrometry. Incidentally, Table 3 also shows the Sb dose (a) in the sample F (semiconductor wafer) obtained by the particle induced X-ray emission analysis in the first step.

TABLE 3
Sb dose in sample F obtained under measuring
conditions (1), (2), (3) of secondary ion
mass spectrometry

| | Measuring | Measuring | Measuring | |
|---|---|---|---|---|
| | condition | condition | condition | |
| | (1) | (2) | (3) | PIXE |
| | b1 | b2 | b3 | a |
| Sb dose | 1.8E14 | 1.1E14 | 1.4E14 | 1.34E14 |
| (atoms/cm²) | | | | |

(*) “PIXE” represents the particle induced X-ray emission analysis.

(Fourth Step)

The dose (a), i.e., 1.3E14 atoms/cm², of Sb in the semiconductor wafer, which was obtained by the particle induced X-ray emission analysis in the first step, was compared by the computer 21 with the doses of Sb shown in Table 3, which were obtained under the measuring conditions 1), 2) and 3) of the secondary ion mass spectrometry in the third step, so as to cause the computer to select the result obtained under the measuring condition 3), which was closest to the result obtained by the particle induced X-ray emission analysis and representing the true dose. Also, the value of {(a−b3/a}, where “a” represents the dose obtained by the particle induced X-ray emission analysis, and “b3” represents the dose obtained under the measuring condition 3) of the secondary ion mass spectrometry, was found to be +0.04 falling within the range from −0.1 to +0.1. In conclusion, it has been found that the measuring condition 3) of the secondary ion mass spectrometry represents the measuring condition that permits measuring the true profile of the concentration in the depth direction or the profile close to the true profile.

Then, the profile of the antimony concentration in the depth direction after a plurality of manufacturing steps such as a plurality of ion implantation steps and a plurality heat treating steps in the research and development of the Si semiconductor device is measured under the measuring condition 3) that permits obtaining the true profile of the antimony concentration in the depth direction. Since the obtained plural profiles of the antimony concentration in the depth direction represents the true antimony profile, it is possible to determine accurately which manufacturing process of the Si semiconductor device is most excellent by comparing and studying the shape of the profile and the antimony dose obtained therefrom. In other words, since the comparison and study of a plurality of manufacturing processes of the Si semiconductor device can be performed accurately in the stage of the research and development, the research and development can be performed efficiently.

Also, the profile of the antimony concentration in the depth direction of the Si semiconductor wafer is measured in the Si semiconductor device factory under the measuring condition 3) that permits obtaining the true profile of the antimony concentration in the depth direction. It is possible to perform accurately the defective analysis of the Si semiconductor device derived from the abnormality in the shape of the antimony profile and the dose by comparing and studying the shapes of the profile of the antimony concentration in the depth direction and the antimony dose derived therefrom in respect of a good sample and a defective sample.

### COMPARATIVE EXAMPLE 3

The operation was performed as in Example 4, except that the first step was not carried out. As shown in Table 3 given previously, the doses obtained under the measuring conditions 1), 2), 3) of the secondary ion mass spectrometry differed from each other. Therefore, it is unclear which value represents the true value or the value close to the true value in the case where the secondary ion mass spectrometry alone was carried out. In other words, it is supported that it is impossible to determine the true profile of the concentration in the depth direction in Comparative Example 3.

It should also be noted that, since the true profile of the Sb concentration in the depth direction cannot be obtained, the research efficiency and the defect analyzing efficiency are deteriorated in the research and development of a Si semiconductor device and in the Si semiconductor device factory.

### EXAMPLE 5

(Sample Preparation Step)

In Example 5, prepared was a sample obtained by introducing ¹¹⁵In (indium) into a surface region of a Si semiconductor wafer by an ion implantation method under an accelerating energy of 20 keV and at a dose of 6.0 E13 cm⁻². The Si semiconductor wafer having the In ions implanted thereinto was cut into pieces each sized at about 1 cm×1 cm by a dicing saw so as to obtain two adjacent samples each sized at 1 cm×1 cm. The size of each sample was measured with a slide gage. The sample was found to have a vertical length of 1.00 cm and a lateral length of 1.00 cm and, thus, the area of the sample was 1.00 cm². Then, these two samples were washed with a pure water so as to remove the cutting dust. One of these samples (sample G) was used in the first step, and the other sample (sample H) was used in the second step.

(First Step)

The chemical analysis was employed for analyzing In (indium) in the Si semiconductor wafer. It should be noted in this connection that, in order to analyze In (indium) in the semiconductor wafer, e.g., Si semiconductor wafer, by the nuclear reaction analysis requiring a large reaction cross sectional area, it is necessary to increase the energy of the ion beam used for bringing about a nuclear reaction to a level higher than the ordinary level, e.g., not higher than 3 MeV. Therefore, it is difficult to carry out nuclear reaction analysis by using the general purpose nuclear reaction analytical apparatus. Of course, it is possible to employ the nuclear reaction analysis, if a nuclear reaction analytical apparatus capable of generating a high energy ion beam is available. Also, if the Rutherford backscattering spectrometry or the particle induced X-ray emission analysis is employed in the case where the In dose in the semiconductor wafer, e.g., a Si semiconductor wafer, is not higher than the order of E13 cm⁻³, it is possible for the sensitivity to be rendered insufficient. Of course, it is possible to employ without difficulty the Rutherford backscattering spectrometry or the particle induced X-ray emission analysis in the case of the In analysis not lower than about E15 cm⁻². Under the circumstances, employed was the chemical analysis which permits analyzing In with a high precision and at a high accuracy.

Sample G was put in a bottle having an inner volume of 250 ml and made of a fluorocarbon polymers. Then, 20 ml of a high purity acidic mixed solution (high purity nitric acid 1: high purity hydrofluoric acid 1: pure water 1) was poured into the bottle, and the bottle was disposed on a hot plate heated to 160° C. so as to thermally decompose the sample G. Further, the reaction mixture was poured into a beaker having an inner volume of 100 ml and made of a fluorocarbon polymers, and the beaker was disposed on a hot plate heated to 180° C. so as to evaporate the volatile component and, thus, to condense the reaction mixture.

After the condensed reaction mixture was cooled, 0.03 ml of a high purity nitric acid was poured into the beaker so as to dissolve the condensed sample. Then, the reaction mixture was poured into a graduated flask having an inner volume of 50 ml and made of a fluorocarbon polymers, followed by adding water to the reaction mixture to permit the volume of the reaction mixture to be increased to reach the gage mark of the graduated flask.

The amount of In (indium) in the solution was measured by an inductively coupled plasma mass spectrometric apparatus and found to be 7.85 ng. Since the area of the sample G was 1.00 cm², the surface density (dose) using the number of atoms was 6.3E13 atoms/cm². The surface density thus obtained is used as the reference dose.

(Second Step)

In this step, the profile of the ¹¹⁵In concentration in the depth direction of a semiconductor wafer sample (sample H) prepared in the sample preparation step was measured by the quadrupole secondary ion mass spectroscopy.

Specifically, the profiles of the ¹¹⁵In concentration in the depth direction of the sample H were obtained by the method 1) given below by using the secondary ion mass spectrometric apparatus shown in FIG. 2. Incidentally, a reference sample into which ¹¹⁵In was introduced by means of an ion implantation under an accelerating energy of 200 keV and at a dose of 1.0E15 cm⁻² was prepared as a reference sample having a known In concentration, said reference sample being used when the profile of the secondary ions in the depth direction was converted into the profile of the concentration in the depth direction.

1) The ¹¹⁵In⁺ ion concentration was measured by using oxygen primary ions (O₂⁺) having an energy of 1 keV, the incident angle of the oxygen primary ion beam being 0°. Then, the profile of the In concentration in the depth direction was obtained from the profile of the ¹¹⁵In⁺ secondary ions in the depth direction.

In the measuring condition 1) described above, used were the oxygen primary ion (O₂⁺) having an energy of 1 keV and the ¹¹⁵In⁺ secondary ion as the secondary ion because the particular combination permits measuring the In profile with the highest sensitivity. If the energy of the primary ion is not higher than 1 keV, it is possible to perform the measurement with a high resolution in the depth direction.

FIG. 7 is a graph showing the profiles of the ¹¹⁵In concentration in the depth direction of the sample H, which were obtained under the measuring condition 1) given above. In the graph of FIG. 7, the depth (nm) of the sample H is plotted on the abscissa, with the ¹¹⁵In concentration (atoms/cm³) being plotted on the ordinate.

(Third Step)

In the first step, the reference dose (a) obtained in the first step was supplied to the computer 21. Then, the profile (b1′) of the In concentration in the depth direction of the sample H (Si semiconductor wafer) obtained in the second step was supplied to the computer, followed by causing the computer to calculate the surface integral in respect of the profile b1′, thereby obtaining the converted dose b1.

Table 4 shows the ¹¹⁵In dose based on the measuring condition 1) of the obtained secondary ion mass spectrometry. Incidentally, Table 4 also shows the ¹¹⁵In dose (a) in the sample G (semiconductor wafer) obtained by the chemical analysis in the first step.

TABLE 4
In dose in sample H obtained under
measuring condition (1) of secondary
ion mass spectrometry

| | Measuring | |
|---|---|---|
| | condition | Chemical |
| | (1) | analysis |
| | b1 | a |
| In dose | 6.21E13 | 6.28E13 |
| (atoms/cm²) | | |

(Fourth Step)

Only one dose of ¹¹⁵In, which is shown in Table 4, was obtained under the measuring condition 1) of the secondary ion mass spectrometry. Therefore, the reference dose (a) was not compared with the converted dose (b1) in part 1 of the fourth step, and the calculation of the formula A given below was performed in part 2 of the fourth step:

X={(a−b1)/a} (A)

where “a” represents the dose measured by the chemical analysis, and “b1” represents the dose measured under the measuring condition 1) of the secondary ion mass spectrometry.

The value of X was found to be 0.01, which falls within the range from −0.1 to +0.1. In other words, it has been found that the measuring condition 1) of the secondary ion mass spectrometry permits measuring the true profile of the concentration in the depth direction or the profile close to the true profile.

The profile of the In concentration in the depth direction after a plurality of manufacturing steps such as a plurality of ion implantation processes and a plurality of heat treating processes in the research and development of the Si semiconductor device is measured under the measuring condition 1) that permits obtaining the true profile of the indium concentration in the depth direction. Since the plural profiles of the indium concentration in the depth direction represents the true indium profile, it is possible to determine accurately which manufacturing process of the Si semiconductor device is most excellent by comparing and studying the shape of the profile, the indium dose obtained therefrom, etc. In other words, since the comparison and study of a plurality of manufacturing processes of a Si semiconductor device can be carried out accurately in the stage of the research and development, the research and development can be carried out efficiently.

Also, the profile of the indium concentration in the depth direction is measured in the factory of the Si semiconductor device under the measuring condition 1) that permits obtaining the true profile of the indium concentration in the depth direction. It is possible to perform accurately the defective analysis of the Si semiconductor device derived from the abnormality in the shape of the profile of the In concentration and the dose by comparing and studying the shapes of the profiles of the In concentration in the depth direction and the In dose derived therefrom in respect of a good sample and a defective sample.

### COMPARATIVE EXAMPLE 4

The operation was performed as in Example 5, except that the first step was not carried out. It is unclear whether or not the dose shown in Table 5, which was obtained under the measuring condition 1) of the secondary ion mass spectrometry, represents the true dose or the dose close to the true dose.

It should also be noted that, since the true profile of the In concentration in the depth direction cannot be obtained, the research efficiency and the defect analyzing efficiency are deteriorated in the research and development of a Si semiconductor device and in the Si semiconductor device factory.

### EXAMPLE 6

The reference dose was measured by the chemical analysis in respect of a Si semiconductor wafer containing Ga as an impurity. Also, a plurality of profiles of the Ga concentration in the depth direction of the semiconductor wafer were obtained by using a secondary ion mass spectrometric apparatus similar to that described previously in conjunction with Example 2. In this secondary ion mass spectrometry, O₂⁺ was used as the primary ion and Ga⁺ was used as the secondary ion. Each profile of the concentration in the depth direction was converted into the dose by the method similar to that described previously in conjunction with Example 2, and the dose thus converted was compared with the reference dose. It has been found that the dose obtained under the measuring condition that the secondary ion was measured with the incident angle of the primary ion beam set at 0° and without blowing an oxygen gas against the semiconductor wafer was closest to the reference dose.

Also, the value of {(a−b)/a}, where “a” represents the reference dose obtained by the chemical analysis, and “b” represents the dose closest to the reference dose, was found to be −0.08, which falls within the range from −0.1 to +0.1. In other words, it has been found that the measuring condition that the secondary ion is measured without blowing an oxygen gas represents the measuring condition that permits obtaining the true profile of the concentration in the depth direction or the profile close to the true profile.

### EXAMPLE 7

The reference dose was measured by the Rutherford backscattering spectrometry (RBS) in respect of a Si semiconductor wafer containing Ge as an impurity. Also, a plurality of profiles of the Ge concentration in the depth direction of the semiconductor wafer were obtained by using a secondary ion mass spectrometric apparatus similar to that described previously in conjunction with Example 1. In this secondary ion mass spectrometry, Cs⁺ was used as the primary ion and GeSi⁻ was used as the secondary ion. Each profile of the concentration in the depth direction was converted into the dose by the method similar to that described previously in conjunction with Example 1, and the dose thus converted was compared with the reference dose. It has been found that the dose obtained under the measuring condition involving the use of (GeSi⁻/Average Si₂⁻) as the secondary ion intensity ratio was closest to the reference dose.

Also, the value of {(a−b)/a}, where “a” represents the reference dose obtained by the Rutherford backscattering spectrometry, and “b” represents the dose obtained under the measuring condition involving the use of (GeSi⁻/Average Si₂⁻) as the secondary ion intensity ratio, was found to be +0.05, which falls within the range from −0.1 to +0.1. In other words, it has been found that the measuring condition involving the use of (GeSi⁻/Average Si₂⁻) as the secondary ion intensity ratio represents the measuring condition that permits obtaining the true profile of the concentration in the depth direction or the profile close to the true profile.

Incidentally, the reference dose was measured by the chemical analysis in place of the Rutherford backscattering spectrometry so as to obtain the result similar to that obtained in Example 7.

### EXAMPLE 8

The reference dose was measured by the nuclear reaction analysis in respect of a Si semiconductor wafer containing F as an impurity. Also, a plurality of profiles of the F concentration in the depth direction of the semiconductor wafer were obtained by using a secondary ion mass spectrometric apparatus similar to that described previously in conjunction with Example 1. In this secondary ion mass spectrometry, Cs⁺ was used as the primary ion and F⁻ or FSi⁻ was used as the secondary ion. Each profile of the concentration in the depth direction was converted into the dose by the method similar to that described previously in conjunction with Example 1, and the dose thus converted was compared with the reference dose. It has been found that the dose obtained under the measuring condition involving the use of (FSi⁻/Average Si₂⁻) as the secondary ion intensity ratio was closest to the reference dose.

Also, the value of {(a−b)/a}, where “a” represents the reference dose obtained by the nuclear reaction analysis, and “b” represents the dose obtained under the measuring condition involving the use of (FSi⁻/Average Si₂⁻) as the secondary ion intensity ratio, was found to be +0.09, which falls within the range from −0.1 to +0.1. In other words, it has been found that the measuring condition involving the use of (FSi⁻/Average Si₂⁻) as the secondary ion intensity ratio represents the measuring condition that permits obtaining the true profile of the concentration in the depth direction or the profile close to the true profile.

Table 5 shows the impurity elements, the measuring conditions for measuring the reference dose, the kinds of the primary ion and the secondary ion used in the secondary ion mass spectrometry, the measuring conditions that permit obtaining the true profile of the concentration in the depth direction, and the values of (a−b)/b in respect of Examples 1 to 8 described above.

TABLE 5

| | Example 1 | Example 2 | Example 3 | Example 4 | Example 5 | Example 6 | Example 7 | Example 8 |
|---|---|---|---|---|---|---|---|---|
| Impurity | As | B | P | Sb | In | Ga | Ge | F |
| elements | | | | | | | | |
| First step | Chemical | NRA | Chemical | PIXE | Chemical | Chemical | RBS or | NRA |
| (Reference | analysis | | analysis | | analysis | analysis | chemical | |
| dose) | | | | | | | analysis | |
| Second step | Primary | Primary | Primary | Primary | Primary | Primary | Primary | Primary |
| (Secondary | ion | ion | ion | ion | ion | ion | ion | ion |
| ion mass | Cs⁺ | O₂⁺ | Cs⁺ | Cs⁺ | O₂⁺ | O₂⁺ | Cs⁺ | Cs⁺ |
| spectrometry) | Secondary | Secondary | Secondary | Secondary | Secondary | Secondary | Secondary | Secondary |
| | ion | ion | ion | ion | ion | ion | ion | ion |
| | AsSi⁻ | B⁺ | PSi⁻ | SbSi⁻ | In⁺ | Ga⁺ | GeSi⁻ | F⁻or |
| | | | | | | | | FSi⁻ |
| Third step | (AsSi⁻/ | B⁺ | (PSi⁻/ | (SbSi⁻/ | In⁺ | Ga⁺ | (GeSi⁻/ | (FSi⁻/ |
| (True profile | Average | | Average | Average | | | Average | Average |
| in depth | Si₂⁻) | | Si₂⁻) | Si₂⁻) | | | Si₂⁻) | Si₂⁻) |
| direction) | | | | | | | | |
| Fourth step | +0.08 | −0.05 | +0.03 | +0.04 | +0.01 | −0.08 | +0.05 | +0.09 |
| (a-b)/a | | | | | | | | |

(*) “RBS” represents the Rutherford backscattering spectrometry.
“NRA” represents the nuclear reaction analysis.
“PIXE” represents the particle induced X-ray emission analysis.

In each of the Examples described above, the reference dose is supplied first to the computer, followed by supplying the profile of the concentration in the depth direction to the computer and subsequently converting the profile of the concentration in the depth direction into the dose. However, the inputting order of the data to the computer is not limited to the order described above in the present invention. For example, it is possible to supply first the profile of the concentration in the depth direction to the computer, followed by converting the profile of the concentration in the depth direction into the dose and subsequently supplying the reference dose to the computer.

Also, in each of the Examples described above, the profiles of the concentration in the depth direction obtained by a plurality of methods are stored in advance in the computer. However, it is also possible to prepare a measuring program that comprises supplying the profiles of the concentration in the depth direction obtained by a plurality of methods to the computer.

As described above in detail, the present invention provides a method for measuring the impurity profile in a semiconductor wafer and a program for measuring the impurity profile in a semiconductor wafer. According to the present invention, the profiles of the impurity concentration in the depth direction of the semiconductor wafer are measured by a plurality of measuring methods. What should be noted is that the present invention produces the effect that it is possible to select the profile of the concentration in the depth direction close to the true profile from among a plurality of obtained profiles of the concentration in the depth direction.

What should also be noted is that, according to the present invention directed to the measuring method of the impurity profile in a semiconductor wafer and the measuring program of the impurity profile in a semiconductor wafer, it is possible to obtain a true profile or a profile close to the true profile.

Additional advantages and modifications will readily occur to those skilled in the art. Therefore, the present invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A method of measuring an impurity profile of a semiconductor wafer, comprising:
obtaining a reference dose by measuring a dose of an impurity in a semiconductor wafer by one measuring method selected from the group consisting of a chemical analysis, a nuclear reaction analysis, a Rutherford backscattering spectrometry and a particle induced X-ray emission analysis, said measuring method being selected in accordance with the kind of said impurity;
obtaining a plurality of impurity profiles measured in a direction of depth of the semiconductor wafer by a plurality of measuring methods;
obtaining a plurality of converted doses by converting each of said plurality of impurity profiles measured in the direction of depth into a dose;
selecting a converted dose closest to said reference dose from said plurality of converted doses; and
selecting an impurity profile which gives the selected dose from said plurality of impurity profiles measured in the direction of depth, as the impurity profile of the semiconductor.

2. The method of measuring an impurity profile of a semiconductor wafer according to claim 1, wherein said impurity is at least one element selected from the group consisting of As, P, Sb, B, In, Ge and Ga, and the measuring method of said reference dose is the chemical analysis.

3. The method of measuring an impurity profile of a semiconductor wafer according to claim 1, wherein said impurity is at least one element selected from the group consisting of B and F, and the measuring method of said reference dose is the nuclear reaction analysis.

4. The method of measuring an impurity profile of a semiconductor wafer according to claim 1, wherein said impurity is at least one element selected from the group consisting of As, Sb, In, Ge and Ga, and the measuring method of said reference dose is the chemical analysis or the Rutherford backscattering spectrometry.

5. The method of measuring an impurity profile of a semiconductor wafer according to claim 1, wherein said impurity is at least one element selected from the group consisting of As, Ge, Sb, In and Ga, and the measuring method of said reference dose is the particle induced X-ray emission analysis.

6. The method of measuring an impurity profile of a semiconductor wafer according to claim 1, wherein said impurity includes As and said reference dose is measured by the chemical analysis, said chemical analysis comprising:
decomposing the semiconductor wafer by using nitric acid, hydrofluoric acid and at least one kind of the compound selected from the group consisting of potassium permanganate, potassium periodate and periodic acid so as to obtain a decomposed liquid containing arsenic and at least one element selected from the group consisting of manganese, potassium and iodine;
ionizing the at least one element contained in the decomposed liquid;
removing the ions of the at least one element from the decomposed liquid by using an ion exchange resin; and
subjecting arsenic contained in the decomposed liquid to a mass spectrometry.

7. The method of measuring an impurity profile of a semiconductor wafer according to claim 1, wherein said impurity includes In and said reference dose is measured by the chemical analysis, said chemical analysis comprising:
dissolving the semiconductor wafer in a mixed aqueous solution containing nitric acid and hydrofluoric acid; and
subjecting In contained in the obtained solution to a mass spectrometry.

8. The method of measuring an impurity profile of a semiconductor wafer according to claim 1, wherein said plurality of impurity profiles measured in the direction of depth are obtained by a secondary ion mass spectrometry in which the measuring conditions are made different for each of said plurality of impurity profiles.

9. The method of measuring an impurity profile of a semiconductor wafer according to claim 8, wherein the measuring conditions that are changed in the secondary ion mass spectrometry include at least one kind of the condition selected from the group consisting of the method for converting the number of counts of secondary ions into the impurity concentration (atoms/cm³), the kind of the primary ion, the energy of the primary ion, the incident angle of the primary ion, and the kind of the secondary ion.

10. The method of measuring an impurity profile of a semiconductor wafer according to claim 1, wherein each of said plurality of impurity profiles measured in the direction of depth can be represented by a graph in which the impurity concentration (atoms/cm³) is plotted on the ordinate and the depth (cm) of the semiconductor wafer is plotted on the abscissa, and each of said plurality of converted doses is a value (atoms/cm²) obtained by calculating a surface integral for each of the graphs.

11. The method of measuring an impurity profile of a semiconductor wafer according to claim 1, wherein the measuring said plurality of impurity profiles measured in the direction of depth is carried out simultaneously with the obtaining said reference dose.

12. A method of measuring an impurity profile of a semiconductor wafer, comprising:
obtaining a reference dose by measuring a dose of an impurity in a semiconductor wafer by one measuring method selected from the group consisting of a chemical analysis, a nuclear reaction analysis, a Rutherford backscattering spectrometry and a particle induced X-ray emission analysis, said measuring method being selected in accordance with the kind of said impurity;
obtaining a plurality of impurity profiles measured in a direction of depth of the semiconductor wafer by a plurality of measuring methods;
obtaining a plurality of converted doses by converting each of said plurality of impurity profiles measured in the direction of depth into a dose;
selecting a converted dose closest to said reference dose from said plurality of converted doses;
calculating a value X in formula (1) given below:
X=(a−b)/a (1)
where “a” is the reference dose (atoms/cm²), and “b” is the selected dose (atoms/cm²); and
selecting an impurity profile which gives a selected dose that permits the value of X to fall within a range from −0.1 to 0.1 from said plurality of impurity profiles measured in the direction of depth, as the impurity profile of the semiconductor.

13. The method of measuring an impurity profile of a semiconductor wafer according to claim 12, wherein the selected dose given by the selected impurity profile permits the value of X to fall within a range from −0.05 to 0.05.

14. A method of measuring an impurity profile of a semiconductor wafer, comprising:
obtaining a reference dose by measuring a dose of an impurity in a semiconductor wafer by one measuring method selected from the group consisting of a chemical analysis, a nuclear reaction analysis, a Rutherford backscattering spectrometry and a particle induced X-ray emission analysis, said measuring method being selected in accordance with the kind of said impurity; and
obtaining an impurity profile measured in a direction of depth of the semiconductor wafer, the impurity profile giving a converted dose that satisfies formula (2) given below:
−0.1≦{(a−b)/a}≦0.1 (2)
where “a” is the reference dose (atoms/cm²), and “b” is a converted dose (atoms/cm²) obtained by converting an impurity profile measured in the direction of depth into a dose.

15. The method of measuring an impurity profile of a semiconductor wafer according to claim 14, wherein the converted dose given by the impurity profile satisfies formula (3) given below:
−0.05≦{(a−b)/a}≦0.05 (3)
where “a” is the reference dose (atoms/cm²), and “b” is the converted dose (atoms/cm²).

16. The method of measuring an impurity profile of a semiconductor wafer according to claim 14, wherein the measuring said plurality of impurity profiles measured in the direction of depth is carried out simultaneously with the obtaining said reference dose.

17. A program for measuring an impurity profile of a semiconductor wafer, comprising:
an instruction for supplying to a computer as a reference dose at least one kind of the dose selected from the group consisting of a dose of an impurity in a semiconductor wafer obtained by a chemical analysis, a dose of the impurity in the semiconductor wafer obtained by a nuclear reaction analysis, a dose of the impurity in the semiconductor wafer obtained by a Rutherford backscattering spectrometry, and a dose of the impurity in the semiconductor wafer obtained by a particle induced X-ray emission analysis;
an instruction for causing the computer to convert each of a plurality of impurity profiles measured in a direction of depth of the semiconductor wafer into a dose, said plurality of impurity profiles being obtained by plural methods; and
an instruction for causing the computer to select a converted dose closest to said reference dose from the plurality of converted doses.

18. The program for measuring an impurity profile of a semiconductor wafer according to claim 17, further comprising:
an instruction for causing the computer to calculate the value of X in formula (4) given below:
X=(a−b)/a (4)
where “a” represents the reference dose (atoms/cm²), and “b” represents the selected dose (atoms/cm²); and
an instruction for causing the computer to select an impurity profile which gives a selected dose that permits the value of X to fall within a range from −0.1 to 0.1, from said plurality of impurity profiles measured in the direction of depth.

19. The program for measuring an impurity profile of a semiconductor wafer according to claim 17, wherein said plural methods includes secondary ion mass spectrometric methods differing from each other in the measuring conditions, and the measuring conditions that are changed include at least one kind of the measuring condition selected from the group consisting of the method for converting the number of counts of the secondary ions into the impurity concentration (atoms/cm³), the kind of the primary ion, the energy of the primary ion, the incident angle of the primary ion, and the kind of the secondary ion.

20. A program for measuring an impurity profile of a semiconductor wafer, comprising:
an instruction for supplying to a computer as a reference dose at least one kind of the dose selected from the group consisting of a dose of an impurity in a semiconductor wafer obtained by a chemical analysis, a dose of the impurity in the semiconductor wafer obtained by a nuclear reaction analysis, a dose of the impurity in the semiconductor wafer obtained by a Rutherford backscattering spectrometry, and a dose of the impurity in the semiconductor wafer obtained by a particle induced X-ray emission analysis;
an instruction for causing the computer to convert an impurity profile measured in a direction of depth of the semiconductor wafer into a dose; and
an instruction for causing the computer to calculate a value X in formula (5) given below:
X=(a−b)/a (5)
where “a” is the reference dose (atoms/cm²), and “b” is the converted dose (atoms/cm²); and
an instruction for causing the computer to find an impurity profile which gives a converted dose that allows the value of X to fall within a range from −0.1 to 0.1.
